(19) United States
(12) Patent Application Publication
(10) Pub. No.: US 2025/0073716 A1
(43) Pub. Date: Mar. 6, 2025

(54) Digital Microfluidic Nucleic Acid Detection Chip, Detection Method, and Detection Apparatus

(71) Applicants: Beijing BOE Sensor Technology Co., Ltd., Beijing (CN); BOE Technology Group Co., Ltd., Beijing (CN)

(72) Inventors: Le GU, Beijing (CN); Yingying ZHAO, Beijing (CN); Bolin FAN, Beijing (CN); Yongjia GAO, Beijing (CN); Li YANG, Beijing (CN); Huazhe LIU, Beijing (CN)

(21) Appl. No.: 18/580,632

(22) PCT Filed: Nov. 24, 2022
(86) PCT No.: PCT/CN2022/134112

§ 371 Date: Jan. 19, 2024

(51) Int. Cl. B01L 3/00; B01L 7/00; C12Q 1/6825; C12Q 1/686

(52) U.S. Cl. CPC B01L 3/502792; B01L 3/502715; B01L 7/52; C12Q 1/6825; C12Q 1/686; B01L 2300/0663; B01L 2300/0819; B01L 2300/165; B01L 2400/0427

(57) ABSTRACT

The present disclosure provides a digital microfluidic nucleic acid detection chip, detection method, and detection apparatus. The digital microfluidic nucleic acid detection chip includes: a first substrate and a second substrate assembled with the first substrate, and a cavity formed between the first substrate and the second substrate includes a functional region (AC), which is configured to perform a nucleic acid detection processing on a droplet to be detected and obtain a hybridization color development signal for indicating whether a target gene exists in the droplet to be detected; the first substrate at least includes a plurality of drive units, which are configured to drive the droplet to be detected to move, a volume of the droplet to be detected is 10 μl to 200 μl, and a dimension of a drive unit is 2 mm to 100 mm in a moving direction of the droplet to be detected.

### CROSS-REFERENCE TO RELATED APPLICATION

The present application is a U.S. National Phase Entry of International Application No. PCT/CN2022/134112 having an international filing date of Nov. 24, 2022. The entire contents of the above-identified application are hereby incorporated by reference.

### TECHNICAL FIELD

The present disclosure relates to, but is not limited to, the field of micro-electromechanical technologies, in particular to a digital microfluidic nucleic acid detection chip, detection method, and detection apparatus.

### BACKGROUND

Microfluidic refers to science and technology involved in a system of processing or manipulating tiny fluids (from nanoliter to liter in volume) by using micro-channels (tens to hundreds of micrometers in size), and is a new interdisciplinary subject involving chemistry, fluid physics, microelectronics, new materials, biology, and biomedical engineering. Because of characteristics of miniaturization and integration, etc., a microfluidic apparatus is usually called a microfluidic chip, also known as a lab on a chip and a micro total analysis system. One of important characteristics of microfluidic is that it has unique fluid properties in micro-scale environment, such as laminar flow and droplets. With help of these unique fluid phenomena, microfluidic may achieve micromachining and micro-operation that are difficult to be completed by a series of conventional methods.

At present, microfluidic is considered to have great development potential and wide application prospects in biomedical research.

### SUMMARY

The following is a summary of subject matter described herein in detail. The summary is not intended to limit the protection scope of claims.

In a first aspect, an embodiment of the present disclosure provides a digital microfluidic nucleic acid detection chip, including: a first substrate; and a second substrate, assembled with the first substrate, and a cavity formed between the first substrate and the second substrate includes a functional region, the functional region is configured to perform a nucleic acid detection processing on a droplet to be detected and obtain a hybridization color development signal for indicating whether a target gene exists in the droplet to be detected; the first substrate at least includes a plurality of drive units arranged in an array, the plurality of drive units are configured to drive the droplet to be detected to move, a volume of the droplet to be detected is 10 μl to 200 μl, and a dimension of a drive unit is 2 mm to 100 mm in a moving direction of the droplet to be detected.

In an exemplary embodiment, on a plane parallel to the digital microfluidic nucleic acid detection chip, the first substrate at least includes: an electrode region, a bonding region located on a side of the electrode region in a first direction, and a lead region located on a side of the electrode region in a second direction, wherein the first direction intersects with the second direction; the plurality of drive units are disposed in the electrode region, each of the drive units includes a plurality of control electrodes arranged in an array, the bonding region includes a plurality of bonding pins, the lead region includes a plurality of signal leads, and each bonding pin is respectively connected with control electrodes at a same position in the plurality of drive units through the signal leads.

In an exemplary embodiment, the drive unit includes a plurality of control electrodes forming m electrode rows and n electrode columns, and control electrodes of an i-th row and a j-th column in the plurality of drive units are respectively connected with a same bonding pin through the signal leads, wherein 1≤i≤m, 1≤j≤n, and m and n are positive integers.

In an exemplary embodiment, m is 5 to 50 and n is 5 to 50.

In an exemplary embodiment, a quantity of the signal leads is the same as a quantity of control electrodes in the drive unit.

In an exemplary embodiment, the electrode region further includes a plurality of connection lines, wherein a first end of at least one connection line is respectively connected with the control electrodes at the same position in the plurality of drive units, and a second end of the connection line is connected with a first end of a signal lead after extending to the lead region, and a second end of the signal lead after is connected with the bonding pin after extending to the bonding region.

In an exemplary embodiment, the electrode region further includes a plurality of via groups arranged in an array, wherein each via group includes a plurality of vias arranged in an array, and a first end of at least one connection line is connected with control electrodes at the same position in the plurality of drive units, respectively, through vias at a same position in the plurality of via groups.

In an exemplary embodiment, the via group includes a plurality of vias forming m via rows and n via columns, and a first end of at least one connection line is respectively connected with control electrodes of an i-th row and a j-th column in the plurality of drive units through vias of the i-th row and the j-th column in the plurality of via groups, wherein 1≤i≤m, 1≤j≤n, and m and n are all positive integers.

In an exemplary embodiment, a control electrode includes a first side and a second side oppositely disposed in the first direction and a third side and a fourth side oppositely disposed in the second direction; in the first direction, distances between a plurality of vias in each via row and first sides of corresponding control electrodes are disposed to be gradually increased or gradually decreased; in the second direction, distances between a plurality of vias in each via column and third sides of corresponding control electrodes are equal; and distances between vias at a same position in each via group and first sides of corresponding control electrodes are equal.

In an exemplary embodiment, on a plane perpendicular to the digital microfluidic nucleic acid detection chip, the first substrate includes a first base substrate, a first conductive layer disposed on a side of the first base substrate facing the second substrate, a first insulation layer disposed on a side of the first conductive layer facing the second substrate, a second conductive layer disposed on a side of the first insulation layer facing the second substrate, a dielectric layer disposed on a side of the second conductive layer facing the second substrate, and a first lyophobic layer disposed on a side of the dielectric layer facing the second substrate; and the control electrodes are disposed in the second conductive layer, the connection lines are disposed in the first conductive layer, a via is disposed on the first insulation layer, and a control electrode is connected with a connection line through the via.

In an exemplary embodiment, the signal leads are disposed in the first conductive layer or the second conductive layer.

In an exemplary embodiment, the second substrate includes a second base substrate, a second structural layer disposed on a side of the second base substrate facing the first substrate, and a second lyophobic layer disposed on a side of the second structural layer facing the first substrate.

In an exemplary embodiment, a distance between a surface on a side of the first lyophobic layer close to the second substrate and a surface on a side of the second lyophobic layer close to the first substrate is 2 μm to 2000 μm.

In an exemplary embodiment, an initial contact angle of the droplet to be detected with the first lyophobic layer and the second lyophobic layer is 105° to 120°.

In an exemplary embodiment, the drive unit includes a full-face control electrode or a plurality of control electrodes arranged in an array, and an area of the full-face control electrode is equal to a sum of areas of the plurality of control electrodes arranged in the array.

In an exemplary embodiment, the drive unit includes a plurality of control electrodes, and a dimension of a control electrode is 1.5 mm to 2 mm in a moving direction of the droplet to be detected.

In an exemplary embodiment, the functional region at least includes a nucleic acid extraction region, a nucleic acid amplification region, a nucleic acid detection region, a first communication path for communicating the nucleic acid extraction region and the nucleic acid amplification region, and a second communication path for communicating the nucleic acid amplification region and the nucleic acid detection region; and the plurality of drive units are respectively disposed at positions corresponding to the nucleic acid extraction region, the nucleic acid amplification region, the nucleic acid detection region, the first communication path, and the second communication path; the nucleic acid extraction region is configured to form the droplet to be detected under drive of the plurality of drive units, and extract a nucleic acid to be amplified from the droplet to be detected; the nucleic acid amplification region is configured to perform a polymerase chain reaction on the nucleic acid to be amplified under drive of the plurality of drive units to form an amplification product; and the nucleic acid detection region is configured to perform a hybridization reaction and a color development reaction on the amplification product under drive of the plurality of drive units, and obtain a hybridization color development signal for indicating whether a target gene exists in the droplet to be detected.

In a second aspect, an embodiment of the present disclosure provides a digital microfluidic nucleic acid detection apparatus, which includes a pipetting apparatus, a temperature control apparatus, a magnetic control apparatus, a signal acquisition and processing apparatus, and the digital microfluidic nucleic acid detection chip as described in one or more embodiments above; wherein the pipetting apparatus is configured to transfer a substance to the digital microfluidic nucleic acid detection chip, and the substance includes: a sample solution or a reagent; the temperature control apparatus is configured to provide a set temperature to the digital microfluidic nucleic acid detection chip; the magnetic control apparatus is configured to provide a set magnetic field to the digital microfluidic nucleic acid detection chip; the signal acquisition and processing apparatus is connected with the digital microfluidic nucleic acid detection chip, and is configured to scan and image the hybridization color development signal formed by the digital microfluidic nucleic acid detection chip for indicating whether a target gene exists in a droplet to be detected to obtain a detection image; and analyze and process the detection image to obtain a detection result, and the detection result includes a positive detection result for indicating a target gene exists in the droplet to be detected or a negative detection result for indicating no target gene exists in the droplet to be detected.

In a third aspect, an embodiment of the present disclosure provides a digital microfluidic nucleic acid detection method, which is suitable for the digital microfluidic nucleic acid detection chip described in one or more exemplary embodiments above, and the method includes: forming a droplet to be detected, and performing a nucleic acid detection processing on the droplet to be detected under drive of a plurality of drive units to obtain a hybridization color development signal for indicating whether a target gene exists in the droplet to be detected.

In an exemplary embodiment, the method further includes: acquiring a detection image obtained by scanning and imaging the hybridization color development signal by a signal acquisition and processing apparatus, analyzing and processing the detection image to obtain a detection result, wherein the detection result includes a positive detection result for indicating a target gene exists in the droplet to be detected or a negative detection result for indicating no target gene exists in the droplet to be detected.

Other aspects may be understood upon reading and understanding drawings and detailed description.

### BRIEF DESCRIPTION OF DRAWINGS

Accompanying drawings are intended to provide further understanding of technical solutions of the present disclosure and constitute a part of the specification, are intended to explain the technical solutions of the present disclosure together with embodiments of the present application, and do not constitute limitations on the technical solutions of the present disclosure. Shapes and sizes of various components in the drawings do not reflect actual scales, but are only intended to schematically illustrate contents of the present disclosure.

FIG. 1 is a schematic diagram of a structure of a digital microfluidic nucleic acid detection apparatus according to an exemplary embodiment of the present disclosure.

FIG. 2 is a schematic diagram of a sectional structure of a digital microfluidic nucleic acid detection chip according to an exemplary embodiment of the present disclosure.

FIG. 3 is a schematic diagram of a sectional structure of another digital microfluidic nucleic acid detection chip according to an exemplary embodiment of the present disclosure.

FIG. 4 is a schematic diagram of an arrangement of control electrodes according to an exemplary embodiment of the present disclosure.

FIG. 5 is a schematic diagram of an electrode region arrangement according to an exemplary embodiment of the present disclosure.

FIG. 6 is a schematic diagram of another electrode region arrangement according to an exemplary embodiment of the present disclosure.

FIG. 7 is a schematic diagram of a nucleic acid extraction region according an exemplary embodiment of the present disclosure.

FIG. 8 is a schematic diagram of a nucleic acid amplification region according to an exemplary embodiment of the present disclosure.

FIG. 9 is a schematic diagram of a nucleic acid detection region according to an exemplary embodiment of the present disclosure.

FIG. 10 is a schematic diagram of a probe array according to an exemplary embodiment of the present disclosure.

FIG. 11 is a schematic diagram of a negative detection result according to an exemplary embodiment of the present disclosure.

FIG. 12 is a schematic diagram of a positive detection result according to an exemplary embodiment of the present disclosure.

### DETAILED DESCRIPTION

Exemplary implementation modes of the present disclosure will be described further in detail below with reference to the accompanying drawings and the embodiments. Following embodiments are used for illustrating the present disclosure, but are not intended to limit the scope of the present disclosure. It should be noted that the embodiments in the present application and features in the embodiments may be arbitrarily combined with each other if there is no conflict.

To make objectives, the technical solutions, and advantages of the present disclosure clearer, the embodiments of the present disclosure will be described in detail with reference to the accompanying drawings. It is to be noted that implementation modes may be implemented in a plurality of different forms. Those of ordinary skills in the art may easily understand such a fact that modes and contents may be transformed into various forms without departing from the purpose and scope of the present disclosure. Therefore, the present disclosure should not be explained as being limited to contents described in following implementation modes only. The embodiments in the present disclosure and features in the embodiments may be combined randomly with each other if there is no conflict.

A scale of the drawings in the present disclosure may be used as a reference in an actual process, but is not limited thereto. For example, a width-length ratio of a channel, a thickness and spacing of each film layer, and a width and spacing of each signal line may be adjusted according to actual needs. A quantity of pixels in a display substrate and a quantity of sub-pixels in each pixel are not limited to numbers shown in the drawings. The drawings described in the present disclosure are schematic structural diagrams only, and one mode of the present disclosure is not limited to shapes, numerical values, or the like shown in the drawings.

Ordinal numerals such as “first”, “second”, and “third” in the specification are set to avoid confusion between constituent elements, but not to set a limit in quantity.

In the specification, for convenience, wordings indicating orientation or positional relationships, such as “middle”, “upper”, “lower”, “front”, “back”, “vertical”, “horizontal”, “top”, “bottom”, “inside”, and “outside”, are used for illustrating positional relationships between constituent elements with reference to the drawings, and are merely for facilitating the description of the specification and simplifying the description, rather than indicating or implying that a referred apparatus or element must have a particular orientation and be constructed and operated in the particular orientation. Therefore, they cannot be understood as limitations on the present disclosure. The positional relationships between the constituent elements may be changed as appropriate according to directions for describing various constituent elements. Therefore, appropriate replacements may be made according to situations without being limited to the wordings described in the specification.

In the specification, unless otherwise specified and defined explicitly, terms “mount”, “mutually connect”, and “connect” should be understood in a broad sense. For example, it may be a fixed connection, or a detachable connection, or an integrated connection. It may be a mechanical connection or an electrical connection. It may be a direct mutual connection, or an indirect connection through middleware, or internal communication between two elements. Those of ordinary skills in the art may understand meanings of these terms in the present disclosure according to specific situations.

In the specification, a transistor refers to an element which at least includes three terminals, i.e., a gate electrode, a drain electrode, and a source electrode. The transistor has a channel region between the drain electrode (drain electrode terminal, drain region, or drain) and the source electrode (source electrode terminal, source region, or source), and a current can flow through the drain electrode, the channel region, and the source electrode. It is to be noted that, in the specification, the channel region refers to a region through which the current mainly flows.

In the specification, an “electrical connection” includes a case that constituent elements are connected together through an element with a certain electrical effect. The “element with the certain electrical effect” is not particularly limited as long as electrical signals may be sent and received between the connected constituent elements. Examples of the “element with the certain electrical effect” not only include electrodes and wirings, but also include switching elements such as transistors, resistors, inductors, capacitors, and other elements with various functions, etc.

In the specification, “parallel” refers to a state in which an angle formed by two straight lines is above −10° and below 10°, and thus also includes a state in which the angle is above −5° and below 5°. In addition, “perpendicular” refers to a state in which an angle formed by two straight lines is above 80° and below 100°, and thus also includes a state in which the angle is above 85° and below 95°.

A triangle, rectangle, trapezoid, pentagon, or hexagon, etc. in the specification are not strictly defined, and it may be an approximate triangle, rectangle, trapezoid, pentagon, or hexagon, etc. There may be some small deformation caused by tolerance, and there may be a chamfer, an arc edge, and deformation, etc.

In the present disclosure, “about” refers to that a boundary is not defined so strictly and numerical values within process and measurement error ranges are allowed.

A Micro Total Analysis System (μ-TAS) was first proposed by Manz and Widmer of the Ciba Geigy Company in 1990, and then it has been developed rapidly. A microfluidic chip is a main development direction and a most active frontier field of the micro total analysis system. Its goal is to integrate functions of a whole laboratory, including sampling, dilution, reagent addition, reaction, separation, and detection on a microchip. Compared with a traditional biochemical analysis laboratory, the microfluidic chip has advantages of automation, a fast detection speed, a small volume, and low sample consumption, etc., which will bring about revolutionary changes in science and technology such as biochemical analysis and medical diagnosis. For the earliest developed flow channel type microfluidic chip, since the flow channel type microfluidic chip needs to achieve liquid drive control with help of peripheral micro pumps, micro valves, and complex pipelines, bubbles and a “dead region effect” are easy to exist in a flow channel. Once the flow channel is formed, it can only be used for specific applications and lacks flexibility. These problems restrict wide application of the flow channel type microfluidic chip. In 1993, Berge discovered that a phenomenon of electrowetting on dielectric through experiments, and fully verified a principle and influencing factors of electrowetting on dielectric in achieving droplet manipulation. Since then, a Digital Micro Fluidics (DMF) technology has been developed vigorously.

A principle of Electrowetting on Dielectric (EWOD) is used for a digital microfluidic chip, a droplet is disposed on a surface with a lyophobic layer, with help of an electrowetting effect, wettability between the droplet and the lyophobic layer is changed by applying a voltage to the droplet, so that a pressure difference and asymmetric deformation are generated inside the droplet, thus operation and control of movement of the droplet may be achieved, and the droplet may be moved, mixed, and separated at micron scale. It has a capability of miniaturizing basic functions of a biological laboratory and a chemical laboratory, etc. to a chip of several square centimeters, thus it is also called a Lab on a Chip (LOC), and has advantages of a small size, portability, flexible combination of functions, and a high integration degree, etc. Digital microfluidic is divided into active digital microfluidic and passive digital microfluidic. A main difference between them is that active digital microfluidic is to drive droplets in an array, which may accurately control a droplet at a position to move alone, while passive digital microfluidic is that droplets at all positions are moved or stopped together. An active digital microfluidic technology may achieve independent control of a control electrode by disposing a Thin Film Transistor (TFT), thus achieving accurate control of droplets. In recent years, the digital microfluidic chip, as a new technology of micro liquid operation and control, has shown great development potential and application prospects in the fields of biology, chemistry, or medicine analysis, etc., due to its advantages such as a simple structure, a small required amount of sample and reagent, easiness in integration, parallel processing, and easiness in automation.

A Polymerase Chain Reaction (PCR) is a molecular biological technique for selectively amplifying target Deoxyribonucleic Acid (DNA) fragments in vitro. It may include following three basic stages: (1) denaturation: a double-stranded structure of a target DNA fragment is melted at a high temperature (such as 94° C. or 95° C.) to form a single-stranded structure; (2) annealing: at a low temperature (such as 50° C., 55° C., or 60° C.), a primer and a single strand achieve renaturation combination according to a principle of base complementary pairing; (3) extension: at an appropriate temperature (such as 72° C.) for DNA synthesis by DNA polymerase, the target DNA fragment is used as a template, and the primer is used as a starting point for nucleic acid synthesis to achieve base binding extension along a direction of template DNA. Three basic stages of denaturation, annealing, and extension constitute a cycle, and nucleic acid synthesis and amplification are performed through continuous cycle denaturation-annealing-extension, so that a large number of target DNA fragments are replicated to achieve highly sensitive molecular diagnosis.

Reverse Dot Blot (RDB) is a kind of nucleic acid hybridization technology, usually a material such as nitrocellulose or nylon membrane is used as a solid-phase material. A variety of specific probes are fixed through a spotting machine, and each probe has a site and is marked with a number. Then nucleic acid to be detected (such as a DNA sample) is hybridized with the probes fixed on a membrane, and a non-hybridized DNA sample is washed away, leaving a targeted DNA with a homologous sequence with the probes. Here, since the DNA sample to be detected (usually a product specifically amplified by PCR, biotin labeling is performed in advance at a 5′ end of a PCR primer, so that an amplification product has biotin labeling correspondingly) has a biotin label, a probe point combined with the DNA sample to be detected has a biotin label, and then a hybridization signal may be displayed through corresponding a color development reaction. In this way, a target nucleic acid fragment in the sample to be detected may be detected, and multiple targets may be screened at the same time through one hybridization, which may be applied to genotyping, pathogen detection, tumor research, and other directions. Among them, probes may be divided into DNA probes and Ribonucleic Acid (RNA) probes according to properties of nucleic acids.

One of main challenges in the field of pathogen molecular diagnosis is an ability of “sample in—result out”. How to reduce manual processing and preparation of samples in an early stage and transfer between different processing processes to minimize manual operation and reduce strict requirements on personnel and laboratory environment is very important.

In a traditional pathogenic nucleic acid detection method, it is usually that after collecting samples (including blood samples or throat swabs, etc.) by a sampling personnel, the samples are transported to a strict PCR laboratory for cell lysis and nucleic acid extraction by a professional operator, and then an extracted nucleic acid is manually transferred to a PCR amplification instrument for nucleic acid amplification, and a PCR product is manually transferred to a detection instrument for nucleic acid detection, wherein time for nucleic acid extraction is about 1 hour to 2 hours, time for nucleic acid amplification is about 1 hour to 2 hours, and time for nucleic acid detection is about 1 hour to 2 hours. It may be seen that an overall detection time length is relatively long and operating environment is limited, which makes it impossible to apply it on a large scale. Therefore, some automatic nucleic acid detection schemes have emerged, such as a scheme based on pump-valve continuous fluid microfluidic chip, a scheme based on a cassette microfluidic chip, or a technical scheme of superposition of two technical schemes, etc. However, it still takes about 4 hours to 5 hours to complete complex processes of nucleic acid extraction, purification, amplification, and detection by using the scheme based on the pump-valve continuous fluid microfluidic chip, and a detection efficiency is low; and the cassette microfluidic chip has a low yield, quality control is difficult, and it cannot be controlled in a process, and if there is a problem in use, it is impossible to determine whether the chip is defective in a manufacturing process or a methodology fails.

At present, the technical field of pathogenic nucleic acid detection involves processes of nucleic acid extraction, amplification, and detection of a large-volume droplet (such as microliter), while a current digital microfluidic chip mainly controls a micro-nano volume droplet, which will lead to a relatively low detection efficiency. In view of problems existing in nucleic acid detection at present, it is of great significance to apply a microfluidic technology to the technical field of nucleic acid detection involving a large-volume droplet (such as 100 microliters to 100 nanoliters) to make a detection process fast.

An embodiment of the present disclosure provides a digital microfluidic nucleic acid detection chip, and the digital microfluidic nucleic acid detection chip includes: a first substrate; and a second substrate, assembled with the first substrate, and a cavity formed between the first substrate and the second substrate may include a functional region, the functional region is configured to perform a nucleic acid detection processing on a droplet to be detected and obtain a hybridization color development signal for indicating whether the droplet to be detected has a target gene; the first substrate may at least include a plurality of drive units arranged in an array, the plurality of drive units are configured to drive the droplet to be detected to move, a volume of the droplet to be detected may be 10 μl to 200 μl, and a dimension of a drive unit may be 2 mm to 100 mm in a moving direction of the droplet to be detected.

In this way, for the digital microfluidic nucleic acid detection chip according to the embodiment of the present disclosure, a large-volume droplet with a volume of about 10 μl to 200 μl is controlled by disposing a drive unit with a dimension of about 2 mm to 100 mm, thereby it may be achieved that the microfluidic technology is applied to the technical field of nucleic acid detection involving a large-volume droplet (such as 10 μl to 200 μl), which makes a detection process fast.

Among them, the dimension of the drive unit may refer to a characteristic length of the drive unit along the moving direction of the droplet to be detected. For example, the dimension of the drive unit may be approximately 2000 μm, 2409 μm, 3000 μm, 3512 μm, 3500 μm, 4000 μm, 6000 μm, 6498 μm, 7000 μm, 10000 μm, 15955 μm, 16000 μm, 20000 μm, 22566 μm, 25000 μm, 25230 μm, 35681 μm, 40000 μm, 50462 μm, 60000 μm, 79888 μm, 900000 μm, or 100000 μm, etc. Here, the embodiment of the present disclosure is not limited thereto.

In an exemplary embodiment, a dimension of a control electrode (e.g., an electrowetting on dielectric electrode) in a drive unit may be about 1.5 mm to 2 mm. For example, the dimension of the control electrode (such as the Electrowetting on Dielectric electrode) in the drive unit may be about 1.5 mm, 1.65 mm, 1.75 mm, 1.85 mm, 1.95 mm, or 2 mm, etc. Among them, the dimension of the control electrode can refer to a characteristic length of the control electrode along the moving direction of the droplet to be detected, such as a side length of a square and a long side of a rectangle.

In an exemplary embodiment, a volume of the droplet to be detected may be about 10 μl, 20 μl, 30 μl, 40 μl, 50 μl, 60 μl, 70 μl, 80 μl, 90 μl, 100 μl, 120 μl, 150 μl, 180 μl, or 200 μl, etc. Here, the embodiment of the present disclosure is not limited thereto.

In an exemplary embodiment, the drive unit includes a plurality of control electrodes forming m electrode rows and n electrode columns, and a control electrodes of an i-th row and a j-th column in the plurality of drive units are respectively connected with a same bonding pin through signal leads, wherein 1≤i≤m, 1≤j≤n, and m and n are positive integers.

In an exemplary embodiment, n may be 5 to 50 and m may be 5 to 50. For example, n may be 5 and m may be 5, in this way, 5*5 control electrodes may be divided into one drive unit, and drive units may be periodically arranged according to 1*5. Here, the embodiment of the present disclosure is not limited thereto.

In an exemplary embodiment, a shape of a control electrode may include a circle or a polygon, for example, the polygon may include any one of a square, a rectangle, a diamond, and a hexagon. Here, the embodiment of the present disclosure is not limited thereto.

In an exemplary embodiment, a quantity of the signal leads is the same as a quantity of control electrodes in a drive unit.

In an exemplary embodiment, an electrode region further may include a plurality of connection lines, a first end of at least one connection line is respectively connected with control electrodes at a same position in a plurality of drive units, and a second end of the connection line is connected with a first end of a signal lead after extending to a lead region, and a second end of the signal lead is connected with a bonding pin after extending to a bonding region.

In an exemplary embodiment, a first base substrate, a first structural layer includes a first conductive layer disposed on a side of the first base substrate facing the second substrate, a first insulation layer disposed on a side of the first conductive layer facing the second substrate, a second conductive layer disposed on a side of the first insulation layer facing the second substrate, and a first lyophobic layer disposed on a side of the second conductive layer facing the second substrate; and a control electrode is disposed in the second conductive layer, a connection line is disposed in the second conductive layer, a via is disposed on the first insulation layer, and the control electrode is connected with the connection line through the via.

In an exemplary embodiment, at least one of the plurality of drive units may include a single monolithic electrode or n×m sub-electrodes arranged in an array, and an area of the monolithic electrode is equal to a sum of areas of the n×m sub-electrodes, wherein n is a positive integer greater than 1 and m is a positive integer greater than 1.

In an exemplary embodiment, a cassette thickness of the digital microfluidic nucleic acid detection chip may be about 2 μm to 2000 μm. For example, the cassette thickness of the digital microfluidic nucleic acid detection chip may be about 2 μm, 5 μm, 10 μm, 15 μm, 20 μm, 25 μm, 30 μm, 40 μm, 50 μm, 60 μm, 70 μm, 100 μm, 150 μm, 200 μm, 300 μm, 600 μm, 800 μm, 1000 μm, 1500 μm, or 2000 μm, etc. Among them, the cassette thickness of the digital microfluidic nucleic acid detection chip may refer to a distance between the first substrate and the second substrate, for example, the cassette thickness of the digital microfluidic nucleic acid detection chip refers to a distance between a surface on a side of the first lyophobic layer in the first substrate close to the second substrate and a surface on a side of the second lyophobic layer in the second substrate close to the first substrate. Here, the embodiment of the present disclosure is not limited thereto.

In an exemplary embodiment, an initial contact angle of the droplet to be detected with the first lyophobic layer and the second lyophobic layer may be about 105° to 120°. For example, an initial contact angle between the droplet to be detected and the first lyophobic layer may be about 105°, 110°, 115°, or 120°, etc. For example, an initial contact angle between the droplet to be detected and the second lyophobic layer may be about 105°, 110°, 115°, or 120°, etc. Here, the embodiment of the present disclosure is not limited thereto.

FIG. 1 is a schematic diagram of a structure of a digital microfluidic nucleic acid detection apparatus according to an exemplary embodiment of the present disclosure. As shown in FIG. 1, a digital microfluidic apparatus may at least include a pipetting apparatus 10, a temperature control apparatus 20, a magnetic control apparatus 30, a digital microfluidic nucleic acid detection chip 40, and a signal acquisition and processing apparatus 50. The pipetting apparatus 10 is configured to transfer a sample solution or reagent to the digital microfluidic nucleic acid detection chip 40, and the sample solution may include a droplet to be detected; the temperature control apparatus 20 is configured to provide a set temperature to the digital microfluidic nucleic acid detection chip 40; the magnetic control apparatus 30 is configured to provide a set magnetic field to the digital microfluidic nucleic acid detection chip 40; the digital microfluidic nucleic acid detection chip 40 is configured to perform automatic control on the droplet to be detected to obtain a hybridization color development signal; the signal acquisition and processing apparatus 50 is connected with the digital microfluidic nucleic acid detection chip 40, and is configured to process the hybridization color development signal obtained by the digital microfluidic nucleic acid detection chip 40 and obtain a detection result. The hybridization color development signal is configured to indicate whether a target gene exists in the droplet to be detected, and the detection result is configured as a positive detection result indicating that the droplet to be detected has a target gene or a negative detection result indicating that the droplet to be detected does not have a target gene.

In an exemplary embodiment, performing automatic control on the droplet to be detected may include: performing a nucleic acid detection processing on the droplet to be detected. For example, the performing the nucleic acid detection processing on the droplet to be detected may include: processing the droplet to be detected to form eluted nucleic acid, performing PCR amplification on the eluted nucleic acid to form a specific PCR product, and performing a hybridization color development reaction on the specific PCR product to form nucleic acid after hybridization and color development, etc.

In an exemplary embodiment, processing the obtained hybridization color development signal and obtaining the detection result may include: scanning the hybridization color development signal, obtaining a detection image, and processing and analyzing the obtained detection image. For example, analyzing and processing the detection image may include: performing an image processing on the detection image to form a gray image, determining a preset gray value corresponding to each probe based on the gray image, determining whether a positive spot is detected at a site corresponding to the probe based on whether the gray value corresponding to each probe is greater than the preset gray value; and forming a detection result according to whether a positive spot is detected at a site corresponding to each probe.

In an exemplary embodiment, determining whether the positive spot is detected at the site corresponding to each probe based on whether the gray value corresponding to each probe is greater than the preset gray value may include: for each probe, determining that a positive spot is detected at the site corresponding to the probe if the gray value corresponding to the probe is greater than the preset gray value; and determining that no positive spot is detected at the site corresponding to the probe if the gray value corresponding to the probe is not greater than the preset gray value. For example, the present gray value may be 40. Here, the embodiment of the present disclosure is not limited thereto.

In an exemplary embodiment, taking a case that probes include an internal reference quality control probe, a color development quality control probe, and a plurality of detection probes as an example, obtaining the detection result according to whether the positive spot is detected at the site corresponding to each probe may include: if it is determined that positive spots are detected at both an internal reference site corresponding to the internal reference quality control probe and a color development site corresponding to the color development quality control probe, and no positive spot is detected at subtype sites corresponding to remaining detection probes, then a negative detection result may be obtained, and the negative detection result is configured to indicate that no target gene exists in the droplet to be detected. Or, if it is determined that positive spots are detected at both an internal reference site corresponding to the internal reference quality control probe and a color development site corresponding to the color development quality control probe, and a positive spot is detected at a subtype site corresponding to at least one of the remaining detection probes, then a positive detection result may be obtained, and the positive detection result is configured to indicate that a target gene indicated by the at least one detection probe exists in the droplet to be detected. Or, if it is determined that no positive spot is detected at a color development site corresponding to the color development quality control probe, a color development failure result may be obtained, and the color development failure result is configured to indicate that a color development act in a nucleic acid detection processing of the droplet to be detected fails, so as to prompt a user to perform detection again. Or, if it is determined that no positive spot is detected at a color development site corresponding to the internal reference quality control probe, a hybridization failure result may be obtained, and the hybridization failure result is configured to indicate that a hybridization act in the nucleic acid detection processing of the droplet to be detected fails, so as to prompt the user that the droplet to be detected fails or collection of the droplet to be detected fails, and the droplet to be detected needs to be provided again.

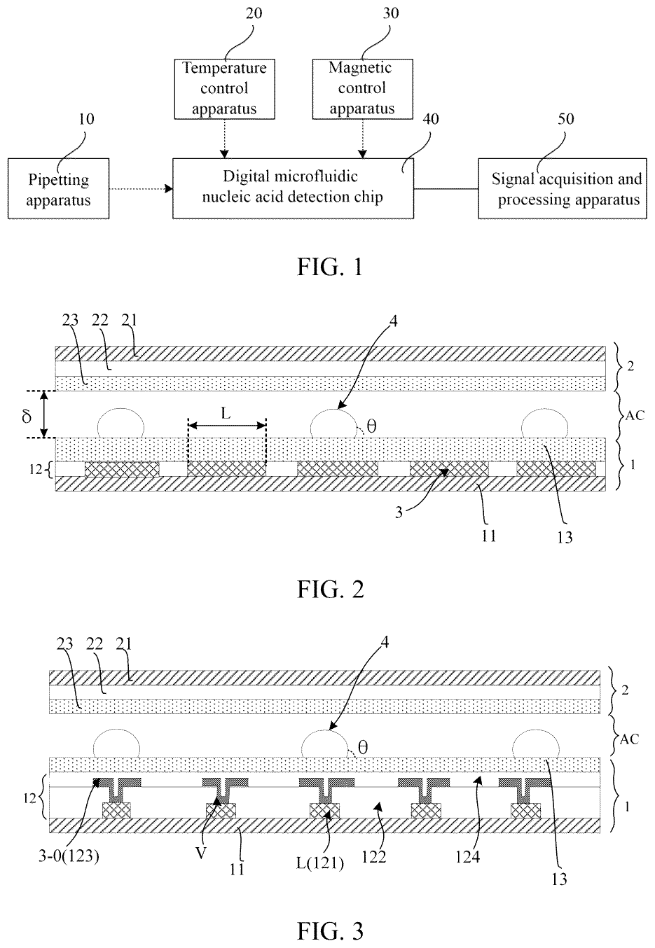

FIG. 2 is a schematic diagram of a sectional structure of a digital microfluidic nucleic acid detection chip according to an exemplary embodiment of the present disclosure. As shown in FIG. 2, in a plane perpendicular to the digital microfluidic nucleic acid detection chip, the digital microfluidic nucleic acid detection chip may include a first substrate 1; and a second substrate 2 assembled with the first substrate 1, the first substrate 1 and the second substrate 2 assembled with the first substrate 1 may be assembled and encapsulated through a sealant, wherein the first substrate 1, the second substrate 2, and the sealant together form a closed cavity, and a droplet 4 to be detected may be disposed in the cavity formed between the first substrate 1 and the second substrate 2. In an exemplary embodiment, the cavity formed between the first substrate 1 and the second substrate 2 may be referred to as a functional region AC, the functional region AC is configured to perform a nucleic acid detection processing on the droplet 4 to be detected and obtain a hybridization color development signal for indicating whether a target gene exists in the droplet to be detected. The first substrate 1 may include a plurality of drive units 3 disposed at positions corresponding to the functional region AC, and is configured to drive the droplet 4 to be detected to move. In a moving direction of the droplet 4 to be detected, a dimension of a drive unit 3 may be about 2 mm to 100 mm to drive the droplet 4 to be detected with a volume of about 10 μl to 200 μl.

In an exemplary embodiment, as shown in FIG. 2, the first substrate 1 may include a first base substrate 11, a first structural layer 12 disposed on a side of the first base substrate 11 facing the second substrate 2, and a first lyophobic layer 13 disposed on a side of the first structural layer 12 facing the second substrate 2, and the second substrate 2 may include a second base substrate 21, a second structural layer 22 disposed on a side of the second base substrate 21 facing the first substrate 1, and a second lyophobic layer 23 disposed on a side of the second structural layer 22 facing the first substrate 1. For example, materials of the first lyophobic layer 13 and the second lyophobic layer 23 may include, but are not limited to, polytetrafluoroethylene (such as Teflon material), fluoropolymer (such as Cytop), and the like, which may enable a droplet to have high surface energy.

In an exemplary embodiment, a cassette thickness δ of the digital microfluidic nucleic acid detection chip may be about 2 μm to 2000 μm. Among them, as shown in FIG. 2, a cassette thickness δ of the digital microfluidic nucleic acid detection chip may refer to a distance between a surface on a side of the first lyophobic layer 13 in the first substrate 1 close to the second substrate 2 and a surface on a side of the second lyophobic layer 23 in the second substrate 2 close to the first substrate 1.

FIG. 3 is a schematic diagram of a sectional structure of another digital microfluidic nucleic acid detection chip according to an exemplary embodiment of the present disclosure. As shown in FIG. 3, the digital microfluidic nucleic acid detection chip may include a first substrate 1; and a second substrate 2 assembled with the first substrate 1. In an exemplary embodiment, a first structural layer 12 may include a first conductive layer 121 disposed on a side of the first base substrate 11 close to the second substrate 2, a first insulation layer 122 disposed on a side of the first conductive layer 121 adjacent to the second substrate 2, and a second conductive layer 123 disposed on a side of the first insulation layer 122 close to the second substrate 2.

In an exemplary embodiment, the first conductive layer 121 and the second conductive layer 123 may be made of a metal material or a transparent conductive material. The metal material may include, but is not limited to, any one or more of Argentum (Ag), Copper (Cu), Aluminum (Al), Titanium (Ti), and Molybdenum (Mo), or an alloy material of the above metals, such as an Aluminum-Neodymium alloy (AlNd) or a Molybdenum-Niobium alloy (MoNb). The transparent conductive material may include Indium Tin Oxide (ITO) or Indium Zinc Oxide (IZO). For example, the first conductive layer 121 and the second conductive layer 123 may be of a single-layer structure or a multi-layer composite structure such as Ti/Al/Ti or ITO/Al/ITO.

In an exemplary embodiment, as shown in FIG. 3, the first conductive layer 121 may include a plurality of connection lines L, the second conductive layer 123 may include a plurality of control electrodes 3-0, and a plurality of vias V may be disposed on the first insulation layer 122, the plurality of control electrodes 3-0 are correspondingly connected with the plurality of connection lines L through the plurality of vias V so as to be connected with signal leads through the plurality of connection lines L.

In an exemplary embodiment, the first conductive layer 121 or the second conductive layer 123 may further include a plurality of signal leads S (not shown in the figure), which are connected with corresponding connection lines L. In this way, the plurality of control electrodes 3-0 may be connected with the plurality of signal leads S through the plurality of vias V and the plurality of connection lines L.

In an exemplary embodiment, the first structural layer 12 may further include a dielectric layer 124 disposed on a side of the second conductive layer 123 close to the second substrate 2 to perform a planarization processing on the first structural layer 12. For example, a material of the dielectric layer may be Polyimide (PI), photoresist (such as SU-8 series), silicon nitride (SiNx), and another material.

In an exemplary embodiment, the second structural layer 22 may include a third conductive layer (not shown in the figure) disposed on a side of the second base substrate 21 close to the first substrate 1. For example, the third conductive layer is configured as a single continuous planar electrode provided with a reference potential. For example, a material of the third conductive layer may be made of a conductive material such as a transparent conductive material or a conductive polymer, the transparent conductive material may include Indium Tin Oxide (ITO) or Indium Zinc Oxide (IZO), and the conductive polymer may include Poly(3,4-ethylenedioxythiophene)/Poly(styrenesulfonate) (Pedot/PSS), etc.

In an exemplary embodiment, the second structural layer 22 may further include a second insulation layer (not shown in the figure) disposed on a side of the third conductive layer close to the first substrate 1, so as to perform a planarization processing on the second structural layer 22.

In an exemplary embodiment, the first insulation layer 122 and the second insulation layer may be made of any one or more of Silicon Oxide (SiOx), Silicon Nitride (SiNx), and Silicon Oxynitride (SiON), and may be a single layer, a multi-layer, or a composite layer.

In an exemplary embodiment, as shown in FIG. 2 and FIG. 3, an initial contact angle θ between the droplet 4 to be detected with a hydrophobic surface (such as the first lyophobic layer 13) may be about 105° to 120°. For example, 0 may generally approach 120 degrees. Here, the embodiment of the present disclosure is not limited thereto.

In an exemplary embodiment, a drive unit may include at least one control electrode 3-0. For example, the drive unit may be a full-face control electrode 3-0 or a plurality of control electrodes 3-0 arranged in an array, and an area of the full-face control electrode 3-0 is equal to a sum of areas of the plurality of control electrodes 3-0 arranged in the array.

In an exemplary embodiment, for the drive unit including a plurality of control electrodes 3-0, a dimension of a control electrode 3-0 may be about 1.5 mm to 2 mm in the moving direction of the droplet 4 to be detected.

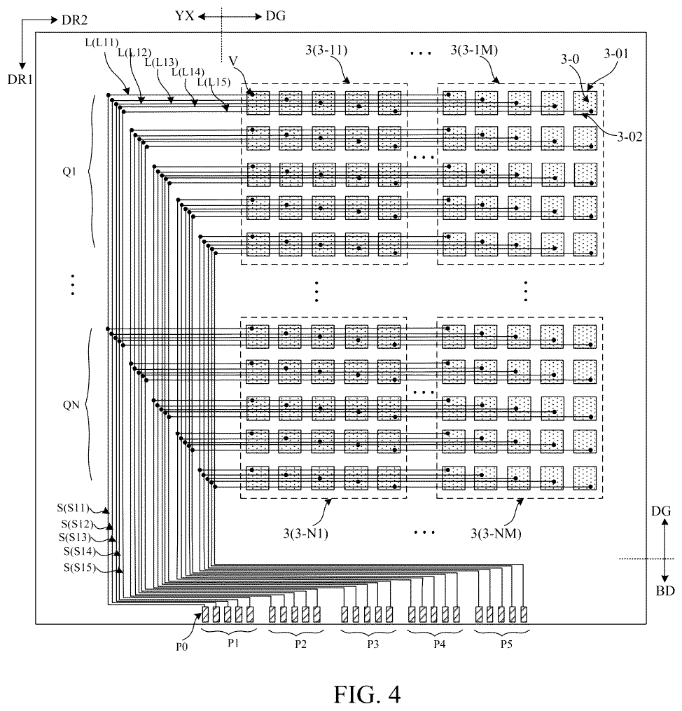

FIG. 4 is a schematic diagram of an arrangement of control electrodes according to an exemplary embodiment of the present disclosure. As shown in FIG. 4, a first substrate may include a plurality of drive units (N×M) arranged in an array, and at least one drive unit may include a plurality of control electrodes (n×m) arranged in an array. For example, taking a case that n is 5 and m is 5 as an example, each drive unit 3 in FIG. 4 includes 5×5 control electrodes 3-0, and drive units 3 are arranged periodically according to N×M.

In an exemplary embodiment, as shown in FIG. 4, on a plane parallel to the digital microfluidic nucleic acid detection chip, the first substrate may at least include an electrode region DJ, a bonding region BD located on a side of the electrode region DJ in a first direction DR1, and a lead region YX located on a side of the electrode region DJ in a second direction DR2, wherein the first direction DR1 intersects with the second direction DR2; a plurality of drive units 3 (for example, a drive unit 3-11 of a first row and a first column to a drive unit 3-NM of an N-th row and an M-th column) are disposed in the electrode region DJ, each drive unit 3 may include a plurality of control electrodes 3-0 arranged in an array, the bonding region BD may include a plurality of bonding pins P0, and the lead region YX may include a plurality of signal leads S, each bonding pin is connected with a control electrode 3-0 at a same position in the plurality of drive units 3 through a signal lead S, respectively.

In an exemplary embodiment, as shown in FIG. 4, the electrode region DG may further include a plurality of connection lines L, wherein a first end of at least one connection line L is respectively connected with a control electrode 3-0 at a same position in the plurality of drive units 3, and a second end of the connection line L is connected with a first end of a signal lead S after extending to the lead region YX, and a second end of the signal lead S is connected with a bonding pin P0 after extending to the bonding region BD.

In an exemplary embodiment, as shown in FIG. 4, control electrodes 3-0 located at a same position in different drive units 3 may share a connection line L (e.g., a first first connection line L11, a first second connection line L12, a first third connection line L13, . . . , or a first fifth connection line L15) to be connected with corresponding peripheral signal leads S (e.g., a first first signal lead S11, a first second signal lead S12, a first third signal lead S13, . . . , or a first fifth signal lead S15). For example, control electrodes 3-0 located in a first row and a first column in the different drive units 3 may share the first first connection line L11 to be connected with the corresponding peripheral first first signal lead S11.

In an exemplary embodiment, a drive unit 3 may include a plurality of control electrodes 3-0 forming m electrode rows and n electrode columns, and control electrodes 3-0 of an i-th row and a j-th column in the plurality of drive units 3 are respectively connected with a same bonding pin P0 through signal leads S, wherein 1≤i≤m, 1≤j≤n, and m and n are positive integers. For example, the control electrodes 3-0 of the i-th row and the j-th column (e.g., control electrodes 3-0 of a first row and a first column) in the plurality of drive units 3 (e.g., a drive unit 3-11 of the first row and the first column, . . . , a drive unit 3-1M of the first row and an M-th column, . . . , a drive unit 3-N1 of an N-th row and the first column, . . . , and a drive unit 3-NM of the N-th row and the M-th column) are respectively connected with a same bonding pin P0 through corresponding signal leads Sij (e.g., the first first signal lead S11).

In an exemplary embodiment, m may be 5 to 50 and n may be 5 to 50. For example, as shown in FIG. 4, m may be 5 and n may be 5.

In an exemplary embodiment, a quantity of signal leads S is the same as a quantity of control electrodes 3-0 in the drive unit 3. For example, as shown in FIG. 4, the drive unit 3 includes 5×5 control electrodes 3-0, that is, a quantity of control electrodes 3-0 in the drive unit 3 is 25, and correspondingly, a quantity of signal leads S is 25.

In an exemplary embodiment, as shown in FIG. 4, the electrode region DG may further include a plurality of via groups arranged in an array, wherein each via group includes a plurality of vias V arranged in an array, and a first end of at least one connection line is connected with control electrodes at a same position in the plurality of drive units respectively, through vias at a same position in the plurality of via groups.

In an exemplary embodiment, as shown in FIG. 4, a via group includes a plurality of vias V forming m via rows and n via columns, and a first end of at least one connection line is respectively connected with control electrodes 3-0 of an i-th row and a j-th column in the plurality of drive units 3 through vias of the i-th row and the j-th column in the plurality of via groups, wherein 1≤i≤m, 1≤j≤n, and m and n are all positive integers.

In an exemplary embodiment, as shown in FIG. 4, a control electrode 3-0 includes a first side 3-01 and a second side 3-02 oppositely disposed in the first direction DR1 and a third side 3-03 and a fourth side 3-04 oppositely disposed in the second direction DR2; in the first direction DR1, distances between a plurality of via V in each via row and first sides 3-01 of corresponding control electrodes 3-0 are disposed to be gradually increased or gradually decreased; in the second direction DR2, distances between a plurality of via V in each via column and third sides 3-03 of corresponding control electrodes 3-0 are equal; and distances between vias V at a same position in each via group and first sides 3-01 of corresponding control electrodes are equal.

In an exemplary embodiment, as shown in FIG. 4, the electrode region DG may include: a plurality of drive units 3 arranged in an array (for example, a first drive unit 3-11 of a first row to an M-th drive unit 3-NM of an N-th row), wherein each of the plurality of drive units 3 may include: a plurality of control electrodes 3-0 arranged in an array; the first substrate 1 may further include n signal lead groups (e.g., a first signal lead group to a fifth signal lead group), wherein each of the signal lead groups may include m signal leads extending along the first direction DR1 (e.g., the first signal lead group includes 5 signal leads S, and the 5 signal leads S include a first first signal lead S11, a first second signal lead S12, a first third signal lead S13, a first fourth signal lead S14, and a first fifth signal lead S15); the plurality of drive units (e.g., the first drive unit 3-11 of the first row to the M-th drive unit 3-NM of the N-th row) may be divided into N drive unit rows (e.g., a first drive unit row Q1 to an N-th drive unit row QN) disposed sequentially along the first direction DR1, wherein each drive unit row may include M drive units disposed sequentially along the second direction DR2 and n connection line groups disposed sequentially along the first direction DR1 (e.g., the first drive unit row Q1 may include: the first drive unit 3-11 of the first row to the M-th drive unit 3-1M of the N-th row, and a first connection line group L11 to a fifth connection line group L15); each drive unit 3 may include n electrode rows disposed sequentially along the first direction DR1, wherein each electrode row may include m drive units 3-0 disposed sequentially along the second direction DR2; each connection line group includes m connection lines extending along the second direction; and for each drive unit row, an i-th connection line group is respectively connected with an i-th electrode row and an i-th signal lead group in the M drive units, and a j-th connection line in the i-th connection line group is respectively connected with a j-th control electrode in the i-th electrode row in the M drive units and a j-th signal lead in the i-th signal lead group, i=1, 2, . . . , n; j=1, 2, . . . , m. In this way, a drive unit is formed with n×m control electrodes, the drive units are periodically arranged by N×M, and for each drive unit row, control electrodes at a same position in different drive units in the drive unit row are connected together through a same connection line, so that the control electrodes at the same position in different drive units in the drive unit row may share a same connection line to be connected to peripheral signal leads, and it may be achieved that for each drive unit, control electrodes at different positions in the drive unit may be independently led to signal leads. In this way, regardless of a quantity of an arrangement period N×M of the drive units, a quantity of peripheral signal leads connected with the control electrodes only depends on a quantity of n×m. Since a quantity of control electrodes involved in the digital microfluidic nucleic acid detection chip is excessive, a quantity of peripheral signal leads may be greatly reduced. For example, taking a case that 5×5 control electrodes are used as one drive unit and the drive units are arranged periodically by 1×5 as an example, a quantity of peripheral signal leads in the digital microfluidic nucleic acid detection chip provided by the exemplary embodiment of the present disclosure may be reduced from 125 to 25, greatly reducing the quantity of peripheral signal leads.

In an exemplary embodiment, as shown in FIG. 4, the electrode region DG may further include M via groups corresponding to the M drive units, and each via group may include n via rows disposed sequentially along the first direction DR1, wherein each via row may include m vias V disposed sequentially along the second direction DR2, and a j-th via in an i-th via row is configured to expose a j-th control electrode in an i-th electrode row; a j-th control electrode in an i-th electrode row in a k-th drive unit is connected with a j-th connection line in an i-th connection line group through a j-th via in an i-th via row in a k-th via group; each control electrode 3-0 may include a first side 3-01 and a second side 3-02 disposed opposite to each other in the first direction DR1, and a third side 3-03 and a fourth side 3-04 disposed opposite to each other in the second direction DR2; in the first direction DR1, distances between m vias in the i-th via row and first sides 3-01 of corresponding control electrodes are disposed to be gradually increased or gradually decreased; in the second direction DR2, distances between n vias in a j-th via column and third sides 3-03 of corresponding control electrodes are equal; and distances between vias at a same position in M via groups in each drive unit row and first sides 3-01 of corresponding control electrodes are equal. i=1, 2, . . . , n; j=1, 2, . . . , m; k=1, 2, . . . , M. In this way, by designing positions of vias, it may be achieved that for every drive unit row, control electrodes at a same position in different drive units in the drive unit row may be share a same connection line to be connected to corresponding signal leads. In this way, regardless of a quantity of an arrangement period N×M of the drive units, a quantity of signal leads only depends on a quantity of n×m. Since a quantity of control electrodes involved in the digital microfluidic nucleic acid detection chip is excessive, a quantity of peripheral signal leads may be greatly reduced.

In an exemplary embodiment, as shown in FIG. 4, the bonding region BD may include n bonding pin groups (e.g., a first bonding pin group P1 to a fifth bonding pin group P5), each of the bonding pin groups may include m bonding pins P0 disposed sequentially along the second direction DR2, and m signal leads in the n signal lead groups are connected with m bonding pins P0 in the n bonding pin groups in one-to-one correspondence. In this way, a drive unit is formed with n×m control electrodes, the drive units are periodically arranged by N×M, for each drive unit row, control electrodes at a same position in different drive units in the drive unit row are connected together through a same connection line, and for each drive unit, control electrodes at different positions in the drive unit are led to different signal leads independently, so that for each drive unit, the control electrodes at different positions in the drive unit are independently led to bonding pins in the bonding region. In this way, a total number (n×m) of bonding pins is the same as a total number (n×m) of control electrodes in one drive unit. In this way, regardless of a quantity of an arrangement period N×M of the drive units, a quantity of peripheral bonding pins only depends on a quantity of n×m. Since a quantity of control electrodes involved in the digital microfluidic nucleic acid detection chip is excessive, a quantity of peripheral bonding pins may be greatly reduced, and drive complexity of a back end may be avoided from being increased. For example, taking a case that 5×5 control electrodes are used as one drive unit, drive units are arranging periodically by 1×5, a quantity of periphery bonding pins may be reduced from 125 to 25, which greatly reduces a quantity of bonding pins.

Among them, N is a positive integer greater than or equal to 1, M is a positive integer greater than 1, n and m are positive integers greater than 1, wherein i=1, 2, . . . , n, j=1, 2, . . . , m; and the second direction DR2 intersects with the first direction DR1.

In an exemplary embodiment, a plurality of bonding pins P0 are sequentially disposed along the second direction DR2, and the second direction DR2 intersects with the first direction DR1.

In an exemplary embodiment, in order to achieve control of a large-volume droplet through a drive unit, a cassette thickness of a digital microfluidic chip and a dimension of the drive unit are matched with a volume of the droplet. For example, taking a case that the drive unit includes a plurality of control electrodes arranged in an array, a volume V of a droplet to be detected may satisfy following formulas.

$$V = πδ {a}^{2} + 2 {π ( \frac{δ}{2 cos θ} )}^{2} [ {af}_{1} ( θ ) + \frac{δ}{2} {f}_{2} ( θ ) ] \\ {f}_{1} ( θ ) = θ - \frac{π}{2} + \frac{sin ( 2 θ - π )}{2} + 2 sin θcos θ \\ {f}_{2} ( θ ) = tan θ [ θ - \frac{π}{2} + \frac{sin ( 2 θ - π )}{2} + 2 cos θ ] - \frac{1}{3} {cos}^{2} θ + {( 1 - sin θ )}^{2}$$

Among them, V represents the volume of the droplet to be detected, θ represents an initial contact angle of the droplet to be detected, δ represents a cassette thickness of a digital microfluidic nucleic acid detection chip, and a represents a radius of a solid-liquid contact surface of the droplet to be detected. For example, the initial contact angle of the droplet to be detected may refer to an initial contact angle between the droplet to be detected and the first lyophobic layer 13, or may refer to an initial contact angle between the droplet to be detected and the second lyophobic layer 23. For example, the cassette thickness δ of the digital microfluidic nucleic acid detection chip may refer to a distance between a surface on a side of the first lyophobic layer 13 close to the second substrate 2 and a surface on a side of the second lyophobic layer 23 close to the first substrate 1.

In an exemplary embodiment, taking a case that the drive unit includes a plurality of control electrodes arranged in an array as an example, the dimension of the drive unit and the cassette thickness of the digital microfluidic nucleic acid detection chip may satisfy a following formula.

$$\frac{L}{δ} = - \sqrt{2} tan θ$$

Among them, θ represents an initial contact angle of the droplet to be detected, θ is generally close to 120°, δ represents the cassette thickness of the digital microfluidic nucleic acid detection chip, and L represents the dimension of the drive unit. For example, the initial contact angle of the droplet to be detected may refer to an initial contact angle between the droplet to be detected and the first lyophobic layer 13, or may refer to an initial contact angle between the droplet to be detected and the second lyophobic layer 23. For example, the cassette thickness δ of the digital microfluidic nucleic acid detection chip may refer to a distance between a surface on a side of the first lyophobic layer 13 close to the second substrate 2 and a surface on a side of the second lyophobic layer 23 close to the first substrate 1. For example, the dimension L of the drive unit may refer to a characteristic dimension of the drive unit in a moving direction of the droplet to be detected. For example, the radius a of the solid-liquid contact surface of the droplet to be detected may be approximately equal to the dimension L of the drive unit.

For example, taking a case that a volume V of the droplet is 10 μl as an example, inventors of the present disclosure obtain verification data shown in the table 1 below through experimental measurements, wherein in the table 1, a unit of the volume V of the droplet is μl, and a unit of the cassette thickness of the drive unit is μm, and a unit of the dimension L of the drive unit is μm.

TABLE 1
Validation data

| Volume | Cassette | | | Dimension L | | | | |
|---|---|---|---|---|---|---|---|---|
| V of the | thickness | | | of the drive | | | | |
| droplet | δ | f₁ (θ) | f₂ (θ) | unit | θ | cos θ | cos² θ | 4 cos² θ |
| 10 | 2000 | 0.027672 | 0.001957 | 2408.66486 | 2.094395 | −0.5 | 0.25 | 1 |
| 10 | 1000 | 0.027672 | 0.001957 | 3512.236455 | 2.094395 | −0.5 | 0.25 | 1 |
| 10 | 300 | 0.027672 | 0.001957 | 6498.064021 | 2.094395 | −0.5 | 0.25 | 1 |
| 10 | 50 | 0.027672 | 0.001957 | 15954.92364 | 2.094395 | −0.5 | 0.25 | 1 |
| 10 | 25 | 0.027672 | 0.001957 | 22566.19967 | 2.094395 | −0.5 | 0.25 | 1 |
| 10 | 20 | 0.027672 | 0.001957 | 25230.2183 | 2.094395 | −0.5 | 0.25 | 1 |
| 10 | 10 | 0.027672 | 0.001957 | 35681.92888 | 2.094395 | −0.5 | 0.25 | 1 |

From analysis of the verification data shown in Table 1 above, it may be seen that in order to achieve application of a microfluidic technology to the field of nucleic acid detection technologies for driving a large-volume droplet (such as 10 μl to 200 μl) and make a detection process fast, relevant parameters of the digital microfluidic nucleic acid detection chip may be set to meet following conditions.
105°≤θ≤120°
10 μl≤V≤200 μl
2 μm≤δ≤2000 μm
2000 μm≤V≤100,000 μm

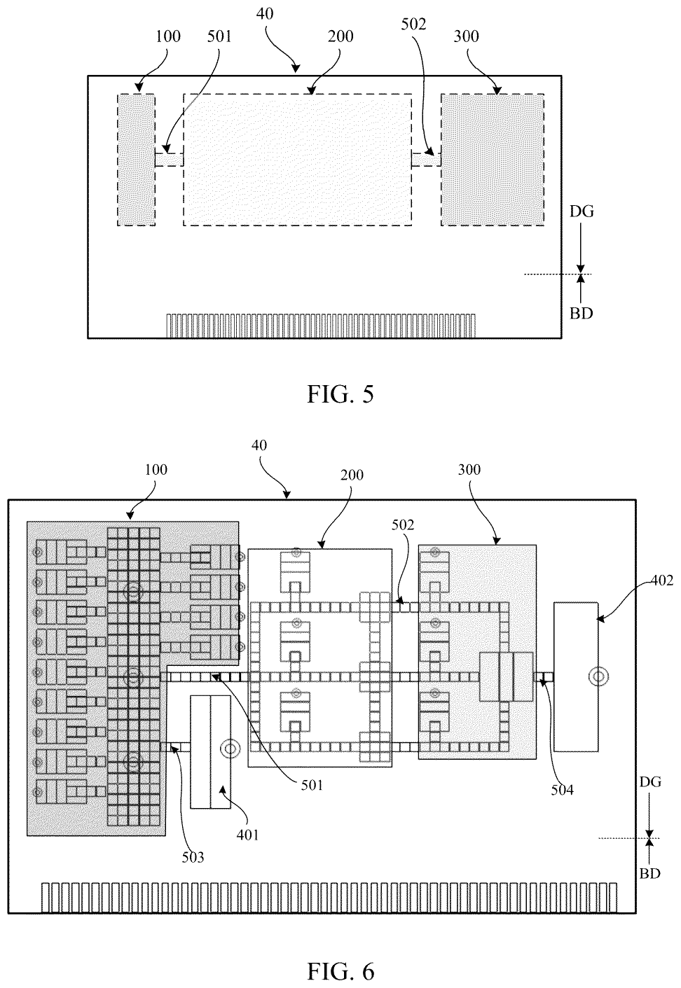

FIG. 5 is a schematic diagram of an electrode region arrangement according to an exemplary embodiment of the present disclosure. As shown in FIG. 5, in a plane parallel to the digital microfluidic nucleic acid detection chip 40, the digital microfluidic nucleic acid detection chip 40 may at least include an electrode region DJ and a bonding region BD located on a side of the electrode region DJ in the first direction DR1.

In an exemplary embodiment, the bonding region BD is configured to be bonded and connected with an external Flexible Printed Circuit (FPC). For example, the bonding region BD may be provided with a bonding pad including a plurality of bonding pins (PINs), and the Flexible Printed Circuit (FPC) may be bound and connected to the pad and configured to transmit a drive signal to a drive unit of the digital microfluidic nucleic acid detection chip 40.

In an exemplary embodiment, for example, the second substrate may be disposed opposite to a functional region on the first substrate and form a closed cavity with the functional region on the first substrate through a sealant, and a plurality of sub-functional regions and communication paths may be formed in the cavity by disposing post spacers.

In an exemplary embodiment, as shown in FIG. 5, the electrode region DJ may include a plurality of sub-functional regions configured to achieve functions such as nucleic acid extraction, amplification, or detection, which may include a nucleic acid extraction region (Nucleic Zone) 100, a nucleic acid amplification region (PCR Zone) 200, and a nucleic acid detection region (Detect Zone) 300, wherein the nucleic acid extraction region 100 and the nucleic acid amplification region 200 are communicated through a first communication path 501, and the nucleic acid detection region 300 and the nucleic acid amplification region 200 are communicated through a second communication path 502. For example, the nucleic acid extraction region 100 is configured to receive a droplet to be detected and a corresponding reagent transferred by an external apparatus (such as the pipetting apparatus 10), process the droplet to be detected to form an eluted nucleic acid, and move the eluted nucleic acid to the nucleic acid amplification region 200 through drive of a drive unit. The nucleic acid amplification region 200 is configured to perform PCR amplification on the eluted nucleic acid to form a specific PCR product and move the specific PCR product to the nucleic acid detection region 300. The nucleic acid detection region 300 is configured to perform a hybridization color development reaction (including a hybridization reaction and a color development reaction) on the specific PCR product through a probe array to form a nucleic acid after hybridization and color development (i.e., a nucleic acid that undergoes a hybridization reaction after color development), to obtain a hybridization color development signal so as to analyze and process the hybridization color development signal to obtain a detection result, and the detection result may include a positive detection result for indicating that a target nucleic acid exists in the droplet to be detected and a negative detection result for indicating that no target nucleic acid exists in the droplet to be detected.

FIG. 6 is a schematic diagram of another electrode region arrangement according to an exemplary embodiment of the present disclosure. As shown in FIG. 6, a plurality of sub-functional regions may further include a first waste solution region 401 and a second waste solution region 402, wherein the nucleic acid extraction region 100 and the first waste solution region 401 are communicated through a third communication path 503, and the nucleic acid detection region 300 and the second waste solution region 402 are communicated through a fourth communication path 504. The first waste solution region 401 is configured to store waste solution formed by processing of the droplet to be detected by the nucleic acid extraction region 100. The second waste solution region 402 is configured to store waste solution formed by processing of the specific PCR product by the nucleic acid detection region 300.

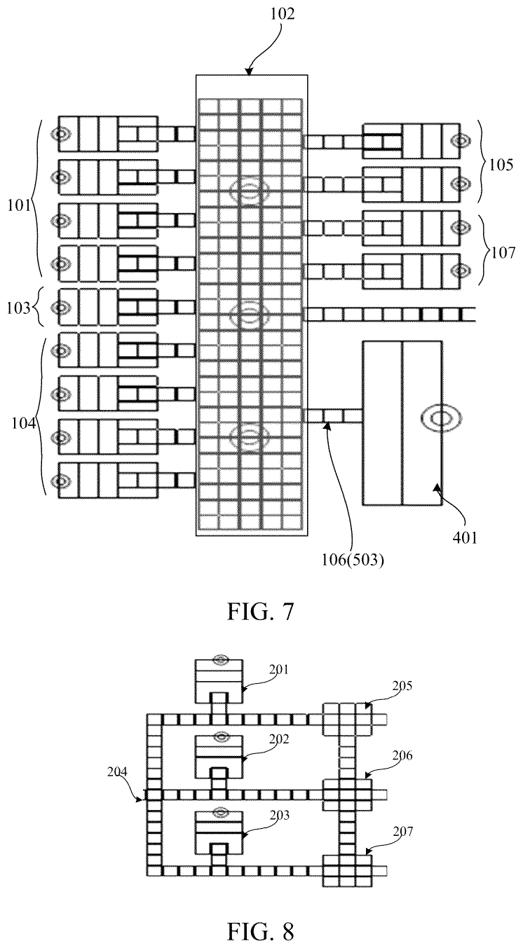

FIG. 7 is a schematic diagram of a nucleic acid extraction region according an exemplary embodiment of the present disclosure. As shown in FIG. 7, the nucleic acid extraction region 100 may include a first washing buffer filling region 101, a mixed incubation region 102, a magnetic bead filling region 103, a second washing buffer filling region 104, an elution filling region 105, a purification channel 106, and an adjuvant filling region 107. The first washing buffer filling region 101, the magnetic bead filling region 103, the second washing buffer filling region 104, the elution filling region 105, the purification channel 106, and the adjuvant filling region 107 are all communicated with the mixed incubation region 102 through a communication path, and the mixed incubation region 102 is communicated with the nucleic acid amplification region 200 through a communication path. Here, the third communication path 503 may serve as the purification channel 106.

In an exemplary embodiment, as shown in FIG. 7, the first washing buffer filling region 101 is configured to provide a first washing buffer, and a corresponding filling hole is disposed on the second substrate where the first washing buffer filling region 101 is located, so that an external apparatus (such as the pipetting apparatus 10) may fill the first washing buffer into the first washing buffer filling region 101. The second washing buffer filling region 104 is configured to provide a second washing buffer, and a corresponding filling hole is disposed on the second substrate where the second washing buffer filling region 104 is located, so that an external apparatus (such as the pipetting apparatus 10) may fill the second washing buffer into the second washing buffer filling region 104. Among them, the second washing buffer and the first washing buffer may be different.

In an exemplary embodiment, as shown in FIG. 7, the magnetic bead filling region 103 is configured to provide magnetic beads.

In an exemplary embodiment, as shown in FIG. 7, the elution filling region 105 is configured to provide elution. A corresponding filling hole is disposed on the second substrate where the elution filling region 105 is located, so that an external apparatus (such as the pipetting apparatus 10) may fill the elution into the elution filling region 105.

In an exemplary embodiment, as shown in FIG. 7, the adjuvant filling region 107 is configured to provide an adjuvant, for example, the adjuvant is a reagent such as Proteinase K that may assist in performing a lysis processing. Among them, the Proteinase K is a powerful proteolytic enzyme with high specific activity, is a reagent for DNA extraction, and can hydrolyze histone combined with a nucleic acid and make DNA free in solution.

In an exemplary embodiment, as shown in FIG. 7, the mixed incubation region 102 may include a sample solution filling sub-region, a mixed lysis sub-region, and a lysis filling sub-region.

In an exemplary embodiment, the sample solution filling sub-region is configured to receive a droplet to be detected provided by an external apparatus (e.g., the pipetting apparatus 10), and a corresponding sample solution filling hole is disposed on the second substrate where the sample solution filling sub-region is located, wherein the sample solution filling hole is configured to enable the external apparatus (e.g., the pipetting apparatus 10) to fill the droplet to be detected into the sample solution filling sub-region 101. For example, sample solution may include, but is not limited to, blood (blood sample), saliva, secretion, urine, or feces, etc., on which nucleic acid detection can be performed. For example, a volume of the sample solution may be about 0.05 mL (milliliter) to 0.2 mL.

In an exemplary embodiment, the lysis filling sub-region is configured to receive lysis solution provided by an external apparatus (e.g., the pipetting apparatus 10), and a corresponding lysis filling hole is disposed on the second substrate where the lysis filling sub-region is located, wherein the lysis filling hole is configured to enable the external apparatus (e.g., the pipetting apparatus 10) to fill the lysis solution into the lysis filling sub-region 101. For example, a volume of the lysis solution may be about 0.2 mL.

In an exemplary embodiment, the mixed lysis sub-region is in communication with the sample solution filling sub-region, the lysis filling sub-region, the first washing buffer filling region 101, the magnetic bead filling region 103, the second washing buffer filling region 104, the elution filling region 105, the purification channel 106, and the adjuvant filling region 107. For example, the mixed lysis sub-region is configured to receive another adjuvant such as Proteinase K provided by the adjuvant filling region 107. The mixed lysis sub-region may further be configured to receive elution provided by the elution filling region 105. The mixed lysis sub-region may further be configured to receive a first washing buffer provided by the first washing buffer filling region 101. The mixed lysis sub-region may further be configured to receive a second washing buffer provided by the second washing buffer filling region 104.

In an exemplary embodiment, the mixed lysis sub-region is configured such that a drive unit of the digital microfluidic nucleic acid detection chip drives the lysis solution to move through an electric field under a temperature (e.g., a constant temperature of 37° C.) provided by the temperature control apparatus 20, so that the droplet to be detected forms a lysed sample solution after being mixed and lysed by the lysis solution. Among them, the lysed sample solution may include a DNA fragment to be detected and other components, such as proteins, lipids, polysaccharides, salt ions, and other cell fragments. The mixed incubation region 102 is further configured such that a drive unit of the digital microfluidic nucleic acid detection chip drives a magnetic bead to move through an electric field, so that the magnetic bead is mixed with the lysed sample solution, and the magnetic bead is combined with the DNA fragment to be detected in the lysed sample solution (i.e., a nucleic acid in a sample) to form a first incubation sample solution; and moreover, in the purification channel 106, the first incubation sample solution is moved along the purification channel 106 under drive of the drive unit, and the peripheral magnetic control apparatus 30 fixes a magnetic bead-DNA mixture in the first incubation sample solution, so that an impurity solution in the first incubation sample solution is discharged from the purification channel 106 under drive of the drive unit and moved to the first waste solution region 401 to form a purified first incubation sample solution, the first incubation sample solution may include a magnetic bead-DNA mixture and an impurity solution, wherein the impurity solution may refer to a solution other than the magnetic bead-DNA mixture, and the purified first incubation sample solution includes a magnetic bead-DNA mixture. Then, the mixed incubation region 102 is further configured such that a drive unit of the digital microfluidic nucleic acid detection chip drives the first washing buffer to move through an electric field, so that the magnetic bead-DNA mixture is mixed and cleaned with the first washing buffer to form a second incubation sample solution; and moreover, in the purification channel 106, the second incubation sample solution is moved along the purification channel 106 under drive of the drive unit, and the peripheral magnetic control apparatus 30 fixes the magnetic bead-DNA mixture after first cleaning in the second incubation sample solution, so that an impurity solution in the second incubation sample solution is discharged from the purification channel 106 and moved to the first waste solution region 401 under e drive of the drive unit to form a purified second incubation sample solution. The second incubation sample solution may include the magnetic bead-DNA mixture after the first cleaning and the impurity solution, wherein the impurity solution may refer to a solution other than the magnetic bead-DNA mixture after the first cleaning, and the purified second incubation sample may include the magnetic bead-DNA mixture after the first cleaning. Thereafter, the mixed incubation region 102 is further configured such that a drive unit of the digital microfluidic nucleic acid detection chip drives the second washing buffer to move through an electric field, so that the magnetic bead-DNA mixture is mixed and cleaned with the second washing buffer to form a third incubation sample solution, and moreover, in the purification channel 106, the third incubation sample solution is moved along the purification channel 106 under drive of the drive unit, and the peripheral magnetic control apparatus 30 fixes a magnetic bead-DNA mixture after second cleaning in the third incubation sample solution, so that an impurity solution in the third incubation sample solution is discharged from the purification channel 106 and moved to the first waste solution region 401 under drive of the drive unit to form a purified third incubation sample solution, the third incubation sample solution may include the magnetic bead-DNA mixture after the second cleaning and the impurity solution, wherein the impurity solution may refer to a solution other than the magnetic bead-DNA mixture after the second cleaning, and the purified second incubation sample solution includes the magnetic bead-DNA mixture after the second cleaning. After that, a mixed lysis sub-region 1022 is configured such that a drive unit of the digital microfluidic nucleic acid detection chip drives the elution to move under a temperature (e.g., a constant temperature of 37° C.) provided by the temperature control apparatus 20, so that the magnetic bead-DNA mixture after the second cleaning is mixed with the elution, and a DNA fragment in the magnetic bead-DNA mixture is eluted through the elution to form a fourth incubation sample solution, and moreover, in the purification channel 106, the peripheral magnetic control apparatus 30 fixes a magnetic bead in the fourth incubation sample solution, and then the drive unit of the digital microfluidic nucleic acid detection chip is driven through an electric field to recycle a supernatant to obtain a DNA fragment to be amplified (i.e., an eluted nucleic acid), wherein the fourth incubation sample solution includes the magnetic bead and the supernatant; and the supernatant includes the DNA fragment to be amplified (i.e., the eluted nucleic acid).

In an exemplary embodiment, as shown in FIG. 7, the purification channel 106 is configured such that the magnetic bead-DNA mixture in the first incubation sample solution is fixed in the purification channel 106 in the nucleic acid extraction region 100 under a magnetic field applied by the peripheral magnetic control apparatus 30, and the impurity solution in the first incubation sample solution is moved to the first waste solution region 401 through drive of the drive unit to form the purified first incubation sample solution. The purification channel 106 is further configured such that the magnetic bead-DNA mixture after the first cleaning in the second incubation sample solution is fixed in the purification channel 106 in the nucleic acid extraction region 100 under a magnetic field applied by the peripheral magnetic control apparatus 30, and the impurity solution in the second incubation sample solution is moved to the first waste solution region 401 through drive of the drive unit to form the purified second incubation sample solution. The purification channel 106 is further configured such that the magnetic bead-DNA mixture after the second cleaning in the third incubation sample solution is fixed in the purification channel 106 in the nucleic acid extraction region 100 under a magnetic field applied by the peripheral magnetic control apparatus 30, and the impurity solution in the third incubation sample solution is moved to the first waste solution region 401 through drive of the drive unit to form the purified third incubation sample solution. The purification channel 106 is further configured such that the magnetic bead in the fourth incubation sample solution is fixed under a magnetic field applied by the peripheral magnetic control apparatus 30. Subsequently, the magnetic field of the magnetic control apparatus 30 is deactivated, so that the drive unit of the digital microfluidic nucleic acid detection chip is driven through an electric field to recycle the supernatant, and the DNA fragment to be amplified (i.e., the eluted nucleic acid) is moved to the nucleic acid amplification region 200.

In the embodiment, the magnetic bead-DNA mixture in the first incubation sample may be fixed in a manner well known in the art. For example, the magnetic control apparatus 30 is disposed in a region where the purification channel 106 is located, and the magnetic control apparatus 30 is controlled to be electrified, and a magnetic field generated by the magnetic control apparatus 30 attracts the magnetic bead-DNA mixture and adsorbs the magnetic bead-DNA mixture on a surface in a cavity. Under a suitable magnetic field, a tiny magnetic bead-DNA mixture converges into a very compact magnet, so will not be taken away by the impurity solution, thus achieving separation of the magnetic bead-DNA mixture and the impurity solution. After the impurity solution is removed, the magnetic control apparatus 30 is controlled to be powered off, the magnetic field disappears, and the magnetic bead-DNA mixture may be moved under the electric field applied by the drive unit.

FIG. 8 is a schematic diagram of a nucleic acid amplification region according to an exemplary embodiment of the present disclosure. As shown in FIG. 8, the nucleic acid amplification region 200 may include a first mixed solution filling sub-region 201, a second mixed solution filling sub-region 202, a third mixed solution filling sub-region 203, a reaction channel 204, a denaturing region 205, an annealing region 206, and an extension region 207, wherein the reaction channel 204 is an annular channel, the first mixed solution filling sub-region 201, the second mixed solution filling sub-region 202, the third mixed solution filling sub-region 203 are communicated with the reaction channel 204, and the denaturing region 205, the annealing region 206, and the extension region 207 are communicated. Corresponding filling holes are respectively disposed on the second substrate where the first mixed solution filling sub-region 201, the second mixed solution filling sub-region 202, and the third mixed solution filling sub-region 203 are located, so that an external apparatus fills an amplification reaction solution-primer mixture into the first mixed solution filling sub-region 201, the second mixed solution filling sub-region 202, and the third mixed solution filling sub-region 203, respectively. For example, the amplification reaction solution-primer mixture may include biotin, primer, deoxy-ribonucleoside triphosphate (dNTP), and an amplification reactant enzyme, etc. After mixing, an amplification program is started. Among them, the primer refers to a known sequence at both ends of a pre-amplified nucleic acid fragment. For example, the amplification reactant enzyme may be DNA polymerase. An amplification reaction solution may also be called a reaction buffer.

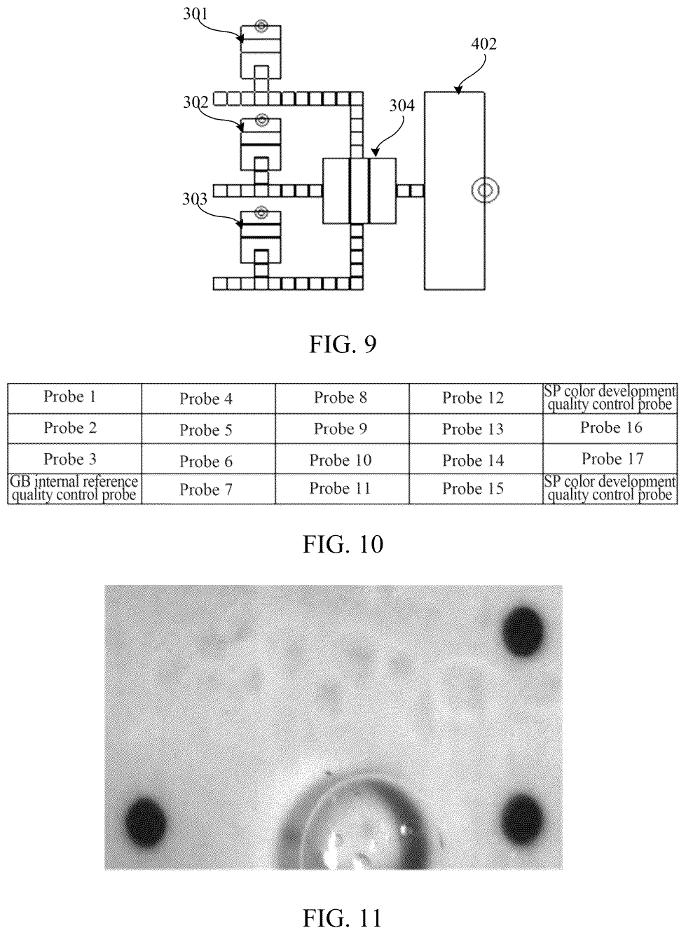

FIG. 9 is a schematic diagram of a nucleic acid detection region according to an exemplary embodiment of the present disclosure. As shown in FIG. 9, the nucleic acid detection region 300 includes a hybridization cleaning solution filling sub-region 301, a membrane processing solution filling sub-region 302, a color development reaction solution filling sub-region 303, and a hybridization membrane region 304, and the hybridization membrane region 304 is communicated with the hybridization cleaning solution filling sub-region 301, the membrane processing solution filling sub-region 302, and the color development reaction solution filling sub-region 303, respectively. In addition, the hybridization membrane region 304 is also communicated with the nucleic acid amplification region 200 and the second waste solution region 402. Corresponding filling holes are disposed on the second substrate where the hybridization cleaning solution filling sub-region 301, the membrane processing solution filling sub-region 302, and the color development reaction solution filling sub-region 303 are located, respectively, so that an external apparatus fills a hybridization cleaning solution, a membrane processing solution, and a color development reaction solution into the hybridization cleaning solution filling sub-region 301, the membrane processing solution filling sub-region 302, and the color development reaction solution filling sub-region 303, respectively. The hybridization membrane region 304 may be configured to perform a hybridization and color development reaction on an amplified nucleic acid and generate a hybridization color development signal for indicating whether a target gene exists in a droplet to be detected.

In an exemplary embodiment, solution extraction holes may be disposed on the second substrate where the first waste solution region 401 and the second waste solution region 402 are located to enable an external apparatus to extract a waste solution.

In an exemplary embodiment, the plurality of drive units 3 are disposed in the first structural layer 12 of the digital microfluidic nucleic acid detection chip 40.

In an exemplary embodiment, the plurality of drive units 3 may be divided into a plurality of drive modules corresponding to the nucleic acid extraction region 100, the nucleic acid amplification region 200, the nucleic acid detection region 300, the first communication path 501, and the second communication path 502, respectively, forming a drive module of the nucleic acid extraction region, a drive module of the nucleic acid amplification region, a drive module of the nucleic acid detection region, a drive module of the first communication path, and a drive module of the second communication path, and each of the drive modules may include at least one drive unit. A working mode of the digital microfluidic nucleic acid detection chip may be as follows: by controlling control electrodes in the drive module of the nucleic acid extraction region, the drive module of the nucleic acid amplification region, the drive module of the nucleic acid detection region, the drive module of the first communication path, and the drive module of the second communication path, a required drive state is provided for a droplet in a corresponding functional region. In an exemplary embodiment, the digital microfluidic nucleic acid detection chip may further include a drive transistor, and the drive transistor is connected with the plurality of drive units 3, and control of the plurality of drive units 3 is achieved through the drive transistor.

In an exemplary embodiment, the pipetting apparatus 10 is configured to add a substance such as a sample solution for forming a droplet, and a reagent for performing a nucleic acid detection processing to a corresponding region of the digital microfluidic nucleic acid detection chip 40. The pipetting apparatus 10 may include a first pipetting sub-module 10-1 corresponding to the nucleic acid extraction region 100, a second pipetting sub-module 10-2 corresponding to the nucleic acid amplification region 200, and a third pipetting sub-module 10-3 corresponding to the nucleic acid detection region 300. The above-mentioned one or more pipetting sub-modules may be disposed on the first substrate 1 or the second substrate 2, corresponding to a corresponding sub-functional region. A sample adding port is disposed in a sub-functional region of the digital microfluidic nucleic acid detection chip 40 corresponding to a pipetting sub-module, and a quantity, a position, and a size of sample adding ports, and types of a sample, a solution, and a reagent injected into a sample adding port of each sub-functional region may be set according to an actual implementation process. The pipetting apparatus 10 is configured to add a desired sample, solution, reagent and so on, to a corresponding sub-functional region through a sample adding port disposed in a sub-functional region (e.g., the nucleic acid extraction region 100, the nucleic acid amplification region 200, and the nucleic acid detection region 300), so as to achieve a corresponding function. In an exemplary embodiment, the pipetting apparatus 10 may be a sample adding pipetting gun or the like.

In an exemplary embodiment, as shown in FIG. 2, the temperature control apparatus 20 may be disposed on a side of the first substrate 1 away from the second substrate 2 or a side of the second substrate 2 away from the first substrate 1, at a position corresponding to a region where at least one sub-functional region in the nucleic acid extraction region 100, the nucleic acid amplification region 200, and the nucleic acid detection region 300 is located, and is configured to provide a set temperature to the nucleic acid extraction region 100, the nucleic acid amplification region 200, and the nucleic acid detection region 300, respectively. In an exemplary embodiment, the temperature control apparatus 20 may include a heater, a temperature sensor, a controller, etc., such as a resistance wire or a semiconductor thermoelectric cooler. The heater forms closed-loop control with the temperature sensor and the controller to accurately and effectively control temperatures of the nucleic acid extraction region 100, the nucleic acid amplification region 200, and the nucleic acid detection region 300.

In an exemplary embodiment, taking a case that the temperature control apparatus 20 provides a set temperature to the nucleic acid extraction region 100 as an example, and the set temperature provided by the temperature control apparatus 20 may be controlled at 37° C.±0.5° C. In an exemplary embodiment, taking a case that the temperature control apparatus 20 provides a set temperature to the nucleic acid amplification region 200 as an example, the set temperature may be controlled between 55° C.±0.5° C. to 95° C.±0.5° C., for example, pre-denaturation at 95° C. for 2 minutes and 45 cyclic PCR amplification cycles are performed, wherein one cyclic amplification cycle may include denaturing at a high temperature of 95° C. for 15 seconds, annealing at a low temperature of 55° C. for 25 seconds, and extending at 72° C. for 15 seconds. Here, the embodiment of the present disclosure is not limited thereto.

In an exemplary embodiment, the temperature control apparatus 20 may include a plurality of sub-modules for implementing a temperature control function, which may include a first temperature control sub-module 20-1 corresponding to the nucleic acid extraction region 100, a second temperature control sub-module 20-2 corresponding to the nucleic acid amplification region 200, and a third temperature control sub-module 20-3 corresponding to the nucleic acid detection region 300. The above-mentioned temperature control sub-modules may be disposed on a side of the first substrate 1 away from the second substrate 2, or disposed on a side of the second substrate 2 away from the first substrate 1, corresponding to corresponding sub-functional regions, and provide suitable temperatures for the corresponding sub-functional regions, respectively.

In an exemplary embodiment, the magnetic control apparatus 30 is configured to generate a magnetic force with a certain field strength, and a droplet may be adsorbed and aggregated and get close to a surface of the digital microfluidic nucleic acid detection chip 40 by using the magnetic control apparatus 30. For example, the magnetic control apparatus 30 may be disposed on a side of the first substrate 1 away from the second substrate 2 or a side of the second substrate 2 away from the first substrate 1, at a position corresponding to a region where the nucleic acid extraction region 100 is located, and is configured to provide a set magnetic field to the nucleic acid extraction region 100. In an exemplary embodiment, the magnetic control apparatus 30 may include a permanent magnet, and a controller, etc. The controller is configured to control a strength of a magnetic field provided to the nucleic acid extraction region 100 by adjusting a distance between the permanent magnet and the first substrate or the second substrate; or the magnetic control apparatus 30 may include an electromagnet, and a controller, etc., and the controller is configured to control a strength of a magnetic field provided to the nucleic acid extraction region 100 by adjusting power on and power off of the electromagnet. Here, the embodiment of the present disclosure is not limited thereto.

In an exemplary embodiment, physically, the temperature control apparatus 20 and the magnetic control apparatus 30 may be disposed separately, or may be combined to form a temperature control and magnetic control integrated apparatus. Here, the embodiment of the present disclosure is not limited thereto.

In an exemplary embodiment, the signal acquisition and processing apparatus 50 may include: a signal acquisition module 50-1 for imaging a hybridization color development signal, and an image processing module 50-2 for processing a detection image, wherein the signal acquisition module 50-1 is disposed at a position corresponding to a region where the nucleic acid detection region 300 in the digital microfluidic nucleic acid detection chip 40 is located, and is configured to scan and image a hybridization color development signal formed by the nucleic acid detection region 300 and used for indicating whether a target gene exists in the droplet to be detected, to obtain a detection image; and the image processing module 50-2 is connected with the signal acquisition module 50-1, and is configured to analyze and process the detection image to obtain a detection result, wherein the detection result may include a positive detection result for indicating a target gene exists in the droplet to be detected or a negative detection result for indicating no target gene exists in the droplet to be detected. In an exemplary embodiment, the signal acquisition module 50-1 may be a Charge-coupled Device (CCD) or the like. In an exemplary embodiment, the image processing module 50-2 may be a processor or the like. In an exemplary embodiment, the signal acquisition module 50-1 and the image processing module 50-2 may be disposed on two sides or on a same side or at other positions of the digital microfluidic nucleic acid detection chip 40, respectively, which are not limited herein. In an exemplary embodiment, a control unit may be integrated in the signal acquisition module 50-1, and the control unit achieves timing control of the drive unit, scanning and imaging timing of the hybridization color development signal, and control timing of a temperature control and magnetic control apparatus and so on in the digital microfluidic nucleic acid detection chip 40.

Taking a case that the digital microfluidic nucleic acid detection chip in the exemplary embodiment of the present disclosure is applied to Human Papilloma Virus (HPV) multiple subtype detection as an example, a detection flow of the digital microfluidic nucleic acid detection chip will be described with reference to structures of sub-functional regions shown in FIG. 5 and FIG. 6.

As shown in FIG. 5 and FIG. 6, the detection flow may include following acts.

(1) In the nucleic acid extraction region 100 of the digital microfluidic nucleic acid detection chip 40, a droplet to be detected is formed from a sample solution to be detected (such as a vaginal secretion) under drive of a drive unit, and then the droplet to be detected is mixed and lysed with a lysis solution and another adjuvant (such as Proteinase K) under a constant temperature condition of 37° C. provided by the temperature control apparatus to form a lysed sample solution. Among them, the lysed sample solution may include a DNA fragment to be detected and other components (such as proteins, lipids, polysaccharides, salt ions, and other cell fragments).

For example, the sample solution to be detected is added to the nucleic acid extraction region 100 of the digital microfluidic nucleic acid detection chip 40 through a filling hole.

For example, the sample solution to be detected includes, but is not limited to, blood, throat swab, vaginal secretion, and feces, etc., and a volume of the sample solution to be detected may be about 0.05 mL to 0.2 mL.

For example, a volume of the droplet to be detected may be about 10 μl to 200 μl.

For example, a volume of the lysis solution may be about 0.2 mL.

(2) In the nucleic acid extraction region 100 of the digital microfluidic nucleic acid detection chip 40, after lysis is completed, the temperature control apparatus 20 stops heating, and a magnetic bead is generated from a solution storage tank, and is combined with the lysed sample solution while being mixed. After combination is completed, a nucleic acid in the lysed sample solution (i.e., the nucleic acid in the droplet to be detected) has been extracted on a surface of the magnetic bead and is transported to a magnetic control region for magnetic adsorption, a waste solution is removed, and a remaining magnetic bead-DNA mixture is adsorbed on a control electrode (such as an Electrowetting on Dielectric electrode) in the drive unit.

For example, a volume of the lysed sample solution may be about 10 μl to 20 μl.

(3) In the nucleic acid extraction region 100 of the digital microfluidic nucleic acid detection chip 40, a first washing buffer is generated from the solution storage tank, the first washing buffer is mixed with the magnetic bead-DNA mixture, then the magnetic bead is released, the first washing buffer is mixed and cleaned with the magnetic bead-DNA mixture and is transported to a magnetic control region for magnetic adsorption under control of the drive unit, and a waste solution is removed to obtain a magnetic bead-DNA mixture after first cleaning. Next, the above cleaning flow is repeated, a second washing buffer is generated from the solution storage tank, the magnetic bead is released, the second washing buffer is mixed and cleaned with the magnetic bead-DNA mixture after the first cleaning and is transported to the magnetic control region for magnetic adsorption under control of the drive unit, and a waste solution is removed to obtain a magnetic bead-DNA mixture after second cleaning.

For example, the first washing buffer with a volume of about 0.05 mL to 0.2 mL is generated from the solution storage tank, the magnetic bead is released, the first washing buffer is mixed and cleaned with the magnetic bead-DNA mixture and is transported to the magnetic control region for magnetic adsorption, and the waste solution is removed. At this time, a magnetic bead-DNA mixture after first cleaning is remained. Next, the second washing buffer with a volume of about 0.05 mL to 0.2 mL is added, and the above cleaning flow is repeated to obtain a magnetic bead-DNA mixture after second cleaning.

(4) In the nucleic acid extraction region 100 of the digital microfluidic nucleic acid detection chip, the cleaned magnetic bead-DNA mixture is mixed with an elution generated in the solution storage tank; after that, the magnetic bead is released, mixed and eluted while being heated; and next, the magnetic bead is adsorbed, and a supernatant is recycled to obtain an eluted nucleic acid.

For example, an elution is generated from the solution storage tank, the cleaned magnetic bead-DNA mixture with a volume of about 10 μL to 60 μL is mixed with the elution, then the magnetic bead is released, mixed and eluted while being heated, then the magnetic bead is adsorbed, and a supernatant is recycled to obtain an eluted nucleic acid.

(5) In the nucleic acid amplification region 200 of the digital microfluidic nucleic acid detection chip 40, an amplification reaction solution-primer mixture with different proportions is designed. After the nucleic acid eluted in a previous act is mixed with the amplification reaction solution-primer mixture with different proportions, an amplification program is started, and PCR amplification is performed at a preset temperature provided by the temperature control apparatus 20 to form an amplified specific PCR product (i.e., an amplified nucleic acid).

For example, a nucleic acid with a volume of about 10 μl to 60 μl eluted in the previous act is added into the amplification reaction solution-primer mixture according to a ratio of 1:1 to 1:3, and the amplification reaction solution-primer mixture may include biotin (labeled, such as fluorescent group), primer, deoxy-ribonucleoside triphosphate (dNTP), and an amplification reactant enzyme, etc. After mixing, an amplification program is started. Among them, the primer refers to a known sequence at both ends of a pre-amplified nucleic acid fragment. For example, the amplification reactant enzyme, which may also be called a reaction buffer, may be DNA polymerase and so on.

For example, parameter settings of the amplification program may be as follows.

An inactivation temperature and time may be 95° C. and 2 min.

A denaturation temperature and time may be 95° C. and 15 s (high temperature denaturation); an annealing temperature and time may be 55° C. and 25 s (low temperature annealing); an extension temperature and time may be 72° C. and 15 s (suitable temperature extension); and a quantity of cycles may be 45.

(6) In the nucleic acid detection region 300 of the digital microfluidic nucleic acid detection chip, a hybridization membrane preprocessing is performed on a hybridization membrane in the hybridization membrane region through a membrane processing solution, and the hybridization membrane in the hybridization membrane region is cleaned through a cleaning solution. After that, the amplified specific PCR product (i.e., the amplified nucleic acid) enters the hybridization membrane region in the nucleic acid detection region 300 for a hybridization reaction under a constant temperature condition provided by the temperature control apparatus 20, and is mixed with a color development reaction solution for a color development reaction to form a fifth incubation sample solution. The hybridization membrane is cleaned by using a hybridization membrane cleaning solution, and a nucleic acid without a hybridization reaction in the fifth incubation sample solution is washed away. Then, the signal acquisition module (such as a CCD) in the signal acquisition and processing apparatus scans and images a hybridization color development signal of a nucleic acid after hybridization and color development (i.e., a nucleic acid with a hybridization reaction after color development) to obtain a detection image, and transmits the detection image to the image processing module for analysis and processing to obtain a detection result. The fifth incubation sample solution includes the nucleic acid after hybridization and color development (i.e., the nucleic acid with a hybridization reaction after color development) and the nucleic acid without a hybridization reaction.

For example, a membrane processing solution with a volume of about 0.05 mL to 0.1 mL is generated from the solution storage tank in the nucleic acid detection region 300 to perform a hybridization membrane preprocessing; the hybridization membrane is cleaned with a cleaning solution with a volume of about 0.05 mL to 0.1 mL generated from the solution storage tank in the nucleic acid detection region 300; the amplified specific PCR product (that is, the amplified nucleic acid) in a previous act is added into the hybridization membrane region, and mixed while being heated, so that the specific PCR product (that is, the amplified nucleic acid) is hybridized with a probe fixed in the hybridization membrane region; after that, the color development reaction solution is added to the hybridization membrane region for a color development reaction, and finally the hybridization membrane is cleaned with a hybridization membrane cleaning solution with a volume of about 0.05 mL to 0.1 mL. The nucleic acid without a hybridization reaction is washed away.

Among them, pipetting is to introduce a series of pre-programmed voltage sequences into the drive unit of the digital microfluidic nucleic acid detection chip, so that a droplet will move on a surface of the chip according to a predetermined path to achieve orderly work.

In actual implementation, an act such as sample adding may be further included. Sample adding is to fill a required sample solution, lysis solution, washing buffer, adjuvant, elution, amplification reaction solution, and primer mixture, etc. into a filling hole in a corresponding sub-functional region of the digital microfluidic nucleic acid detection chip through a pipetting apparatus.

In an exemplary embodiment, the hybridization membrane region in the nucleic acid detection region 300 of the digital microfluidic nucleic acid detection chip is provided with a hybridization membrane, and different probes are spotted on a solid phase membrane through a spotting machine to form the hybridization membrane, i.e., the hybridization membrane region includes a probe array, the probe array may include a plurality of probes arranged in an array, and the plurality of probes may include a color development quality control probe, an internal reference quality control probe, and a probe specifically combined with different types of pathogens, wherein each probe point is different from an adjacent probe point, and different subtypes of pathogens may be detected. For example, FIG. 10 is a schematic diagram of a probe array according to an exemplary embodiment of the present disclosure. As shown in FIG. 10, the probe array may include: 2 SP color development quality control probes, 1 GB internal reference quality control probe, and 17 detection probes specifically combined with different subtypes of HPV, such as probe 1 to probe 17.

FIG. 11 is a schematic diagram of a negative detection result according to an exemplary embodiment of the present disclosure. As shown in FIG. 11, taking a case that a microfluidic chip provided by an embodiment of the present disclosure is applied to detection of multiple subtypes of HPV as an example, an internal reference site and a color development site of a hybridization membrane are positive spots, and no positive spot is detected in remaining HPV subtype target points, then a negative detection result may be obtained, and the negative detection result is configured to indicate that there is no target gene corresponding to different subtypes of HPV in a droplet to be detected.

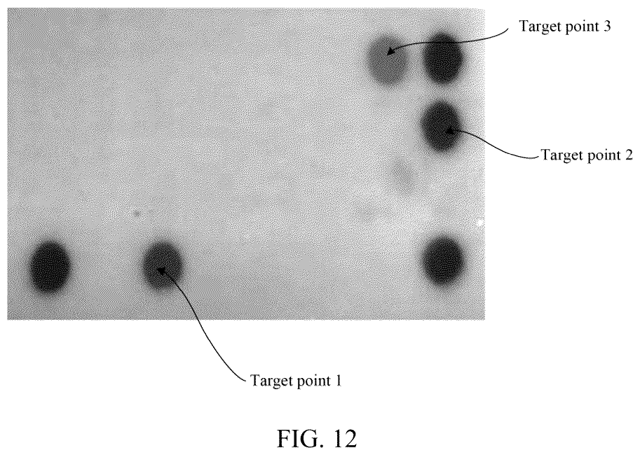

FIG. 12 is a schematic diagram of a positive detection result according to an exemplary embodiment of the present disclosure. As shown in FIG. 12, taking a case that a microfluidic chip provided by an embodiment of the present disclosure is applied to detection of multiple subtypes of HPV as an example, and taking HPV6, HPV16, and HPV31 subtypes as targets, positive spots are also detected in HPV6, HPV16, and HPV31 target points in addition that positive spots are detected at an internal reference site corresponding to a GB internal reference quality control probe and a color development site corresponding to an SP color development quality control probe, and a detection image obtained by scanning of a CCD will show a gray value, so a positive detection result may be obtained, and the positive detection result is configured to indicate existence of target genes of HPV6, HPV16, and HPV31 subtypes in a droplet to be detected. For example, as shown in Table 2 below, in the detection image obtained by scanning of the CCD, a gray value corresponding to HPV6 target point is 111.80, a gray value corresponding to HPV16 target point is 135.05, and a gray value corresponding to HPV31 target point is 81.20.

In addition, if no positive spot is detected at a color development site corresponding to an SP color development quality control probe, a color development failure result may be obtained, and the color development failure result is configured to indicate that a color development act in a nucleic acid detection processing of the droplet to be detected fails, so as to prompt a user to re-detect.

In addition, if no positive spot is detected at an internal reference site corresponding to a GB internal reference quality control probe, a hybridization failure result may be obtained, and the hybridization failure result is configured to indicate that a hybridization act in a nucleic acid detection processing of the droplet to be detected fails, so as to prompt a user that the droplet to be detected fails or collection of the droplet to be detected fails, and the droplet to be detected needs to be re-provided.

TABLE 2
Interpretation of pathogen detection results

| | HPV probe | Gray value |
|---|---|---|
| Target point 1 | HPV6 | 111.80 |
| Target point 2 | HPV16 | 135.05 |
| Target point 3 | HPV31 | 81.20 |

Based on a technical concept of the embodiment of the present disclosure, an embodiment of the present disclosure also provides a digital microfluidic nucleic acid detection method, which is suitable for the digital microfluidic nucleic acid detection chip in one or more of the above embodiments. A digital microfluidic nucleic acid detection method in an exemplary embodiment of the present disclosure may include following acts.

S1, a droplet to be detected is formed.

S2, under drive of a plurality of drive units, a nucleic acid detection processing is performed on the droplet to be detected, and a hybridization color development signal for indicating whether a target gene exists in the droplet to be detected is obtained.

In an exemplary embodiment, the digital microfluidic nucleic acid detection method in the exemplary embodiment of the present disclosure may further include following acts.

S3, a detection image obtained by scanning and imaging the hybridization color development signal by a signal acquisition and processing apparatus is acquired.

S4, the detection image is analyzed and processed to obtain a detection result, wherein the detection result includes a positive detection result for indicating a target gene exists in the droplet to be detected or a negative detection result for indicating no target gene exists in the droplet to be detected.

The above description of the embodiment of the digital microfluidic nucleic acid detection method is similar to that of the above embodiments of the digital microfluidic nucleic acid detection chip and apparatus, and the embodiment of the digital microfluidic nucleic acid detection method has similar beneficial effects as the embodiments of the digital microfluidic nucleic acid detection chip and apparatus. For technical details undisclosed in the embodiment of the digital microfluidic nucleic acid detection method of the present disclosure, those skilled in the art should understand with reference to the description in the embodiments of the digital microfluidic nucleic acid detection chip and apparatus of the present disclosure, and the technical details will not be repeated here.

Although the implementation modes disclosed in the present disclosure are as above, the described contents are only implementation modes used for convenience of understanding the present disclosure and are not intended to limit the present disclosure. Any skilled person in the art to which the present disclosure pertains may make any modification and alteration in forms and details of implementation without departing from the spirit and scope of the present disclosure. However, the patent protection scope of the present disclosure should be subject to the scope defined in the appended claims.

1. A digital microfluidic nucleic acid detection chip, comprising: a first substrate; and a second substrate, assembled with the first substrate, wherein a cavity formed between the first substrate and the second substrate comprises a functional region, the functional region is configured to perform a nucleic acid detection processing on a droplet to be detected and obtain a hybridization color development signal for indicating whether a target gene exist in the droplet to be detected; the first substrate at least comprises a plurality of drive units arranged in an array, the plurality of drive units are configured to drive the droplet to be detected to move, a volume of the droplet to be detected is 10 μl to 200 μl, and a dimension of a drive unit is 2 mm to 100 mm in a moving direction of the droplet to be detected.

2. The digital microfluidic nucleic acid detection chip according to claim 1, wherein on a plane parallel to the digital microfluidic nucleic acid detection chip, the first substrate at least comprises: an electrode region, a bonding region located on a side of the electrode region in a first direction, and a lead region located on a side of the electrode region in a second direction, wherein the first direction intersects with the second direction; the plurality of drive units are disposed in the electrode region, each of the drive units comprises a plurality of control electrodes arranged in an array, the bonding region comprises a plurality of bonding pins, the lead region comprises a plurality of signal leads, and each bonding pin is respectively connected with control electrodes at a same position in the plurality of drive units through the signal leads.

3. The digital microfluidic nucleic acid detection chip according to claim 2, wherein the drive unit comprises a plurality of control electrodes forming m electrode rows and n electrode columns, and control electrodes of an i-th row and a j-th column in the plurality of drive units are respectively connected with a same bonding pin through the signal leads, wherein 1≤i≤m, 1≤j≤n, and m and n are positive integers.

4. The digital microfluidic nucleic acid detection chip according to claim 3, wherein m is 5 to 50 and n is 5 to 50.

5. The digital microfluidic nucleic acid detection chip according to claim 2, wherein a quantity of the signal leads is the same as a quantity of control electrodes in the drive unit.

6. The digital microfluidic nucleic acid detection chip according to claim 2, wherein the electrode region further comprises a plurality of connection lines, a first end of at least one connection line is respectively connected with the control electrodes at the same position in the plurality of drive units, and a second end of the connection line is connected with a first end of a signal lead after extending to the lead region, and a second end of the signal lead is connected with the bonding pin after extending to the bonding region.

7. The digital microfluidic nucleic acid detection chip according to claim 6, wherein the electrode region further comprises a plurality of via groups arranged in an array, each via group comprises a plurality of vias arranged in an array, and a first end of at least one connection line is connected with the control electrodes at the same position in the plurality of drive units, respectively, through vias at a same position in the plurality of via groups.

8. The digital microfluidic nucleic acid detection chip according to claim 7, wherein the via group comprises a plurality of vias forming m via rows and n via columns, and a first end of at least one connection line is respectively connected with control electrodes of an i-th row and a j-th column in the plurality of drive units through vias of the i-th row and the j-th column in the plurality of via groups, 1≤i≤m, 1≤j≤n, and m and n are all positive integers.

9. The digital microfluidic nucleic acid detection chip according to claim 8, wherein a control electrode comprises a first side and a second side oppositely disposed in the first direction, and a third side and a fourth side oppositely disposed in the second direction; in the first direction, distances between a plurality of vias in each via row and first sides of corresponding control electrode are disposed to be gradually increased or gradually decreased; in the second direction, distances between a plurality of vias in each via column and third sides of corresponding control electrode are equal; and distances between vias at a same position in each via group and first sides of corresponding control electrode are equal.

10. The digital microfluidic nucleic acid detection chip according to claim 6, wherein on a plane perpendicular to the digital microfluidic nucleic acid detection chip, the first substrate comprises: a first base substrate, a first conductive layer disposed on a side of the first base substrate facing the second substrate, a first insulation layer disposed on a side of the first conductive layer facing the second substrate, a second conductive layer disposed on a side of the first insulation layer facing the second substrate, and a first lyophobic layer disposed on a side of the second conductive layer facing the second substrate; and the control electrodes are disposed in the second conductive layer, the connection lines are disposed in the first conductive layer, a via is disposed on the first insulation layer, and a control electrode is connected with a connection line through the via.

11. The digital microfluidic nucleic acid detection chip according to claim 10, wherein the signal leads are disposed in the first conductive layer or the second conductive layer.

12. The digital microfluidic nucleic acid detection chip according to claim 10, wherein the second substrate comprises a second base substrate, a second structural layer disposed on a side of the second base substrate facing the first substrate, and a second lyophobic layer disposed on a side of the second structural layer facing the first substrate.

13. The digital microfluidic nucleic acid detection chip according to claim 12, wherein a distance between a surface on a side of the first lyophobic layer close to the second substrate and a surface on a side of the second lyophobic layer close to the first substrate is 2 μm to 2000 μm.

14. The digital microfluidic nucleic acid detection chip according to claim 12, wherein an initial contact angle between the droplet to be detected with at least one of the first lyophobic layer and the second lyophobic layer is 105° to 120°.

15. The digital microfluidic nucleic acid detection chip according to claim 1, wherein the drive unit comprises a full-face control electrode or a plurality of control electrodes arranged in an array, and an area of the full-face control electrode is equal to a sum of areas of the plurality of control electrodes arranged in the array.

16. The digital microfluidic nucleic acid detection chip according to claim 1, wherein the drive unit comprises a plurality of control electrodes and a dimension of a control electrode is 1.5 mm to 2 mm in a moving direction of the droplet to be detected.

17. The digital microfluidic nucleic acid detection chip according to claim 1, wherein the functional region at least comprises: a nucleic acid extraction region, a nucleic acid amplification region, a nucleic acid detection region, a first communication path for communicating the nucleic acid extraction region and the nucleic acid amplification region, and a second communication path for communicating the nucleic acid amplification region and the nucleic acid detection region;
the nucleic acid extraction region is configured to form the droplet to be detected under drive of the plurality of drive units, and extract a nucleic acid to be amplified from the droplet to be detected;
the nucleic acid amplification region is configured to perform a polymerase chain reaction on the nucleic acid to be amplified under drive of the plurality of drive units to form an amplification product; and
the nucleic acid detection region is configured to perform a hybridization reaction and a color development reaction on the amplification product under drive of the plurality of drive units, and obtain a hybridization color development signal for indicating whether a target gene exists in the droplet to be detected.

18. A digital microfluidic nucleic acid detection apparatus, comprising: a pipetting apparatus, a temperature control apparatus, a magnetic control apparatus, a signal acquisition and processing apparatus, and a digital microfluidic nucleic acid detection chip according to claim 1; wherein
the pipetting apparatus is configured to transfer a substance to the digital microfluidic nucleic acid detection chip, and the substance comprises: a sample solution or a reagent;
the temperature control apparatus is configured to provide a set temperature to the digital microfluidic nucleic acid detection chip;
the magnetic control apparatus is configured to provide a set magnetic field to the digital microfluidic nucleic acid detection chip;
the signal acquisition and processing apparatus is connected with the digital microfluidic nucleic acid detection chip, and is configured to scan and image the hybridization color development signal formed by the digital microfluidic nucleic acid detection chip for indicating whether a target gene exists in a droplet to be detected to obtain a detection image; and analyze and process the detection image to obtain a detection result, and the detection result comprises a positive detection result for indicating a target gene exists in the droplet to be detected or a negative detection result for indicating no target gene exists in the droplet to be detected.

19. A digital microfluidic nucleic acid detection method using a digital microfluidic nucleic acid detection chip according to claim 1, comprising:
forming a droplet to be detected; and
performing a nucleic acid detection processing on the droplet to be detected under drive of a plurality of drive units to obtain a hybridization color development signal for indicating whether a target gene exists in the droplet to be detected.

20. The digital microfluidic nucleic acid detection method according to claim 19, wherein the method further comprises:
acquiring a detection image obtained by scanning and imaging the hybridization color development signal by a signal acquisition and processing apparatus; and
analyzing and processing the detection image to obtain a detection result, wherein the detection result comprises a positive detection result for indicating a target gene exists in the droplet to be detected or a negative detection result for indicating no target gene exists in the droplet to be detected.
